SEQUENCE LISTING (5 sequences)

SEQ ID NO: 1 - DNA, 21 bp, Artificial Sequence
  Synthetic construct
ggctgggcctgcaagtccgcg

SEQ ID NO: 2 - DNA, 21 bp, Artificial Sequence
  Synthetic construct
gcacttggcacacttgaacca

SEQ ID NO: 3 - DNA, 21 bp, Artificial Sequence
  Synthetic construct
gacggaggctaagcgtcgcaa

SEQ ID NO: 4 - DNA, 83 bp, Artificial Sequence
  Synthetic construct
aatgatacggcgaccaccgagatctacacacactctttccctacacgacgctcttccgatctttccaagagccaggcccgtac

SEQ ID NO: 5 - DNA, 83 bp, Artificial Sequence
  Synthetic construct
caagcagaagacggcatacgagatcactgtgtgactggagttcagacgtgtgctcttccgatcgagccccattttgaaacacc

(19) United States
(12) Patent Application Publication
(10) Pub. No.: US 2022/0010307 A1
(43) Pub. Date: Jan. 13, 2022

(54) COMPOSITIONS AND METHODS FOR MODULATING TRANSCRIPTIONAL ACTIVITY OF AMPLIFIED ONCOGENES CONTAINED ON EXTRACHROMOSOMAL DNA

(71) Applicants: The Regents of the University of California, Oakland, CA (US); Ludwig Institute for Cancer Research Ltd, La Jolla, CA (US); The Board of Trustees of the Leland Stanford Junior University, Stanford, CA (US)

(72) Inventors: Paul Mischel, La Jolla, CA (US); Bing Ren, La Jolla, CA (US); Kristen Turner, La Jolla, CA (US); Sihan Wu, La Jolla, CA (US); Vineet Bafna, Oakland, CA (US); Nam-phuong Nguyen, Oakland, CA (US); Howard Chang, Stanford, CA (US)

(21) Appl. No.: 17/311,980

(22) PCT Filed: Dec. 10, 2019
(86) PCT No.: PCT/US19/65515

§ 371 Date: Jun. 8, 2021

Related U.S. Application Data
(60) Provisional application No. 62/777,686, filed on Dec. 10, 2018.
(60) Provisional application No. 62/938,161, filed on Nov. 20, 2019.

(51) Int. Cl. C12N 15/11; C12N 15/113; A61K 31/7105; A61K 45/06

(52) U.S. Cl. CPC C12N 15/111; C12N 15/113; A61K 31/7105; C12N 2320/31; C12N 2310/20; C12N 2310/11; A61K 45/06

(57) ABSTRACT

Provided herein are, inter alia, methods and compositions to detect, monitor and treat cancer, wherein the cancer includes amplified extrachromosomal oncogenes. The methods are useful for personalized treatment and exploit differential expression and chromatin structure of extrachromosomal oncogenes in cancer cells.

### CROSS-REFERENCES TO RELATED APPLICATIONS

This application claims the benefit of U.S. Provisional Application No. 62/777,686 filed on Dec. 10, 2018, and U.S. Provisional Application No. 62/938,161, filed on Nov. 20, 2019, which are incorporated herein by reference in entirety and for all purposes.

### GOVERNMENT LICENSE RIGHTS

This invention was made with government support under CA009523, D023527, GM114362, NS73831, NS80939, HG007735 and CA209919 awarded by the National Institutes of Health and under DBI1458557 and IIS1318386 awarded by the National Science Foundation. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048537-621001W0 SEQUENCE LISTING ST25.TXT, created on Dec. 10, 2019, 1,632 bytes, machine format IBM-PC, MS Windows operating system, is hereby incorporated by reference.

### BACKGROUND

Oncogenes are commonly amplified on extrachromosomal DNA particles (ecDNA) in cancer, but our understanding of the structure of ecDNA and its impact on gene regulation is limited. For example, it has yet to be confirmed the ecDNA is truly circular because of the typically short reads obtained by Next Generation Sequencing. Furthermore, it is unknown whether high level expression of oncogenes originates predominantly from chromosomal or extrachromosomal DNA. Finally, the chromatin structure of ecDNA is not known. How chromatin is organized (compaction) determines its relative transcriptional activity.

There is a need in the art for the targeted treatment of ecDNA cancers and personalized treatment methods that make use of the differential expression of extrachromosomal DNA in cancer cells. The methods and compositions provided herein, inter alia, address these and other needs in the art.

### BRIEF SUMMARY OF THE INVENTION

In one aspect is provided a method of detecting an amplified extrachromosomal oncogene in a human subject in need thereof, the method including: (i) obtaining a biological sample from a human subject; (ii) detecting whether an amplified extrachromosomal oncogene is present in the sample by contacting the biological sample with an oncogene-binding agent and detecting binding between the amplified extrachromosomal oncogene and the oncogene-binding agent.

In another aspect is provided a method of treating cancer in a subject in need thereof, the method including: (i) obtaining a biological sample from a human subject; (ii) detecting whether an amplified extrachromosomal oncogene is present in the sample by contacting the biological sample with an oncogene-binding agent and detecting binding between the amplified extrachromosomal oncogene and the oncogene-binding agent; and (iii) administering to the human subject an effective amount of an anti-cancer agent.

In another aspect is provided a method of detecting an amplified extrachromosomal oncogene in a cancer subject undergoing treatment for cancer, the method including: (i) obtaining a first biological sample from the cancer subject undergoing treatment for cancer; and (ii) detecting in the first biological sample a first level of an amplified extrachromosomal oncogene.

In another aspect is provided an extrachromosomal nucleic acid protein complex including an extrachromosomal cancer-specific nucleic acid bound to an endonuclease through an extrachromosomal cancer-specific nucleic acid binding RNA.

In another aspect is provided a method for inducing apoptosis in a cancer cell, the method including: (i)contacting a cancer cell with an effective amount of an extrachromosomal cancer-specific nucleic acid binding RNA bound to an endonuclease; (ii) allowing the extrachromosomal cancer-specific nucleic acid binding RNA to hybridize to an extrachromosomal cancer-specific nucleic acid, thereby binding the endonuclease to the extrachromosomal cancer-specific nucleic acid; and (iii) allowing the endonuclease to cleave the extrachromosomal cancer-specific nucleic acid, thereby inducing apoptosis in the cancer cell.

### BRIEF DESCRIPTION OF THE DRAWINGS

FIGS. 1A-B show that ecDNA physical structure is circular. FIG. 1A shows a cartoon of global workflow to characterize the structure and function of ecDNA. FIG. 1B shows a composite breakpoint graph generated by AmpliconArchitect, in silico digestion map and the assembled contig from BioNano optical mapping of GBM39 ecDNA. Arrows indicate breakpoints connected by discordant paired-end WGS reads.

FIGS. 2A-F shows ecDNA drives high levels of RNA expression. FIG. 2A shows a cartoon of workflow of an RNA-seq data analysis. FIG. 2B is a graph showing ecDNA gene expression within the transcriptome of GBM39 cells. Red dots: genes on ecDNA (circular amplification). FIG. 2C is a violin plot showing ecDNA gene expression in one TCGA-GBM sample (red data points) compared to noncircular genes in the TCGA-GBM cohort (violin and box plot distribution, N=36 biologically independent samples). FIG. 2D is a violin plot showing Z-score of the gene expression plotted in FIG. 2C, with Z-scores plotted as +1. FIG. 2E is a graph showing allele-specific gene copy number and mRNA expression level in GBM39 cells (circular amplified region (ecDNA) is highlighted). FIG. 2F is a violin plot showing gene copy number comparing circular and linear amplifications (8068 circular and 6247 linear amplified genes from 77 samples, two-sided Wilcoxon test).

FIGS. 3A-B present data showing an example of chromatin landscape of ecDNA. FIG. 3A is a graph showing global and long (>1 Kb) ATAC-seq fragment size distribution of ecDNA and chrDNA (110 ecDNA and 1,571 chrDNA long fragments. Two-sided KS test. N=2 biologically independent samples, showing one of the representative result.). FIG. 3B is a bar graph showing ATAC-seq peak number per 10 Kb in GBM39 cells (Circular, 714 windows; Linear, 268 windows; Random, 313,762 windows. N=2 biologically independent samples. Kruskal-Wallis test.).

FIGS. 4A-C present data showing characterization of ecDNA structure by whole genome sequencing. FIG. 4A is a box plot showing ecDNA number per metaphase in GBM39, COLO320DM and PC3 cell line. Boxplots show median, upper and lower quartiles; whiskers indicate 1.5× interquartile range (at least 20 metaphase spreads from 3 biologically independent samples were counted). FIG. 4B is a graph and microscopy pictures showing Representative linear amplicon breakpoint graph in GBM39 cells (left), with FISH validation of its chromosomal loci (scale bar: left, 10 μm; right, 5 μm). These imaging experiments were repeated at least for 3 times, with each replicate showing similar results. FIG. 4C is a dot plot showing the size and copy number of 41 reconstructed circular structures in 37 cancer cell lines.

FIG. 5 shows a pipeline to integrate whole genome sequencing and BioNano optical mapping, in order to characterize ecDNA structure.

FIGS. 6A-B present data showing that genes on ecDNA are highly expressed. FIG. 6A is a graph showing the transcript levels of transcriptome in the U87 GBM cell line, which lacks ecDNA. Green data points represent the same genes that are found on ecDNA in the GBM39 cell line. FIG. 6B is a panel of graphs showing the ecDNA gene expression levels within the transcriptome of COLO320DM and PC3 cells, and selected TCGA samples. Red dots represent genes located on ecDNA (circular amplification genes). For both FIGS. 6A-B, FPKM: fragments per kilobase of transcript per million mapped reads.

FIGS. 7A-B present data showing identified histone modifications on ecDNA. FIG. 7A shows H3K4me1 and H3K27ac ChIP-seq results in cycling GBM39 cells. Zoom-in demonstrates the ecDNA region. FIG. 7B is a bar graph showing the results of imaging experiments aimed at the quantification of H3K9me3 and H3K27me3 foci per ecDNA in GBM39 cells in metaphase; all imaging experiments were repeated at least for 3 times, with each replicate showing similar results.

FIGS. 8A-F present data showing that circularization of ecDNA can enable novel DNA interaction. FIG. 8A shows a possible model depicting local and distal interactions with EGFR promoter and proposed model for CRISPRi masking of EGFR promoter. FIG. 8B shows another possible model depicting local and distal interactions with EGFR promoter and proposed model for CRISPRi masking of EGFR promoter. FIG. 8C and FIG. 8D are graphs showing qPCR quantifications of gene expression in regions proximal and distal to EGFR. For both FIGS. 8C and 8D, data are mean±s.e.m.; n=3; each data point represents three technical replicates from one representative result (criNC: CRISPRi negative control; One-way ANOVA; N.S, not significant; **, P<0.01; ***, P<0.001; ****, P<0.0001). FIG. 8E is a picture of a western blot showing exogenous expression of EGFRvIII in U87 cells (U87-EGFRvIII) and the activation of EGFR signaling was confirmed by western blot; this experiment was repeated for 3 times, with each replicate showing similar results. FIG. 8F is a graph showing the results of qPCR quantification of EGFR-neighboring gene expression in U87 cells, with and without ectopic EGFRvIII overexpression; for this figure the data are mean±s.e.m.; n=3; each data point represents three technical replicates from one representative result (Welch's t-test; N.S, not significant; GBAS, P=0.038; EGFR, P=0.003).

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The term “small molecule” as used herein refers to a low molecular weight organic compound that may regulate a biological process. In embodiments, small molecules are drugs. In embodiments, small molecules have a molecular weight less than 900 daltons. In embodiments, small molecules are of a size on the order of one nanometer.

The term “organic compound” as used herein refers to any of a large class of chemical compounds in which one or more atoms of carbon are covalently linked to atoms of other elements.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbonemodified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements is contiguous or non-contiguous.

The term “recombination” as used herein refers to a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (e.g., the one that experienced the double-strand break), and leads to the transfer of genetic information from the donor to the target. Homology-directed repair may result in an alteration of the sequence of the target molecule (e.g. insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA.

The term “non-homologous end joining (NHEJ)” refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

“Nuclease” and “endonuclease” are used interchangeably herein to mean an enzyme which possesses endonucleolytic catalytic activity for polynucleotide cleavage. The term includes site-specific endonucleases such as, designer zinc fingers, transcription activator-like effectors (TALEs), homing meganucleases, and site-specific endonucleases of clustered, regularly interspaced, short palindromic repeat (CRISPR) systems such as, e.g., Cas proteins.

A “ribonucleoprotein complex,” or “ribonucleoprotein particle” as provided herein refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. Non-limiting examples of ribonucleoproteins include ribosomes, telomerase, RNAseP, hnRNP, CRISPR associated protein 9 (Cas9) and small nuclear RNPs (snRNPs). The ribonucleoprotein may be an enzyme. In embodiments, the ribonucleoprotein is an endonuclease. Thus, in embodiments, the ribonucleoprotein complex includes an endonuclease and a ribonucleic acid. In embodiments, the endonuclease is a CRISPR associated protein 9.

The term “Kruppel-associated box” or “KRAB” as used herein refers to a category of transcriptional repression domains, which typically consists of about 75 amino acid residues.

The term “topologically associating domain” or “TAD” as used herein refers to a self-interacting genomic region wherein DNA sequences physically interact with each other more frequently than with sequences outside the TAD. These three dimensional chromosome structures are present in animals as well as some plants, fungi, and bacteria.

The term “site-specific modifying enzyme” or “RNA-binding site-specific modifying enzyme” as used herein a polypeptide that binds RNA and is targeted to a specific DNA sequence, such as a Cas9 polypeptide. A site-specific modifying enzyme as described herein is targeted to a specific DNA sequence by the RNA molecule to which it is bound. The RNA molecule includes a sequence that binds, hybridizes to, or is complementary to a target sequence within the target DNA, thus targeting the bound polypeptide to a specific location within the target DNA (the target sequence). This RNA molecule can be a small guide RNA (sgRNA). In some cases, the sgRNAs can be selected to inhibit transcription of target loci (e.g., targeted to optimized human CRISPRi target sites), activate transcription of target loci (e.g., targeted to optimized human CRISPRa target sites. In other instances, the Cas9 protein can be a nuclease deficient sgRNA-mediated nuclease (dCas9). This dCas9 can also comprise a dCas9 domain fused to a transcriptional modulator. This transcriptional modulator can be, e.g., a DNA methyltransferase.

By “cleavage” it is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In some embodiments, a complex comprising a guide RNA and a site-specific modifying enzyme is used for targeted double-stranded DNA cleavage.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The term “EGFR” or “EGFR protein” as provided herein includes any of the recombinant or naturally-occurring forms of the epidermal growth factor receptor (EGFR) or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR. In embodiments, EGFR is the protein as identified by the NCBI sequence reference GI: 29725609, homolog or functional fragment thereof.

The term “c-Myc” as provided herein includes any of the recombinant or naturally-occurring forms of the cancer Myelocytomatosis (c-Myc) or variants or homologs thereof that maintain c-Myc activity (e.g. within at least 50%, 80%, 90%, 95%, 96%, 97%, 98%, 99% or 100% activity compared to c-Myc). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring c-Myc. In embodiments, c-Myc is the protein as identified by Accession No. Q6LBK7, homolog or functional fragment thereof.

The terms “N-Myc” as provided herein includes any of the recombinant or naturally-occurring forms of the N-myc proto-oncogene protein (N-Myc) or variants or homologs thereof that maintain N-Myc activity (e.g. within at least 50%, 80%, 90%, 95%, 96%, 97%, 98%, 99% or 100% activity compared to N-Myc). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring N-Myc. In embodiments, N-Myc is the protein as identified by Accession No. P04198, homolog or functional fragment thereof.

The terms “cyclin D1” as provided herein includes any of the recombinant or naturally-occurring forms of the cyclin D1 protein (cyclin D1) or variants or homologs thereof that maintain cyclin D1 activity (e.g. within at least 50%, 80%, 90%, 95%, 96%, 97%, 98%, 99% or 100% activity compared to cyclin D1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cyclin D1. In embodiments, cyclin D1 is the protein as identified by Accession No. P24385, homolog or functional fragment thereof.

The terms “ErbB2”, or “erythroblastic oncogene B,” as provided herein includes any of the recombinant or naturally-occurring forms of the receptor tyrosine-protein kinase erbB-2 (ErbB2) or variants or homologs thereof that maintain ErbB2activity (e.g. within at least 50%, 80%, 90%, 95%, 96%, 97%, 98%, 99% or 100% activity compared to ErbB2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ErbB2. In embodiments, ErbB2 is the protein as identified by Accession No. P04626, homolog or functional fragment thereof.

The terms “CDK4”, or “cyclin-dependent kinase 4” as provided herein includes any of the recombinant or naturally-occurring forms of the cyclin dependent kinase 4 (CDK4) or variants or homologs thereof that maintain CDK4activity (e.g. within at least 50%, 80%, 90%, 95%, 96%, 97%, 98%, 99% or 100% activity compared to CDK4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDK4. In embodiments, CDK4is the protein as identified by Accession No. P11802, homolog or functional fragment thereof.

The terms “CDK6”, or “cyclin-dependent kinase 6” as provided herein includes any of the recombinant or naturally-occurring forms of the cyclin dependent kinase 6 (CDK6) or variants or homologs thereof that maintain CDK6activity (e.g. within at least 50%, 80%, 90%, 95%, 96%, 97%, 98%, 99% or 100% activity compared to CDK6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDK6. In embodiments, CDK6 is the protein as identified by Accession No. Q00534, homolog or functional fragment thereof.

The terms “BRAF” as provided herein includes any of the recombinant or naturally-occurring forms of the serine/threonine-protein kinase B-Raf (BRAF) or variants or homologs thereof that maintain BRAF activity (e.g. within at least 50%, 80%, 90%, 95%, 96%, 97%, 98%, 99% or 100% activity compared to BRAF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BRAF. In embodiments, BRAF is the protein as identified by Accession No. P15056, homolog or functional fragment thereof.

The terms “MDM2”, or “mouse double minute 2” as provided herein includes any of the recombinant or naturally-occurring forms of the mouse double minute 2 homolog (MDM2) or variants or homologs thereof that maintain MDM2 activity (e.g. within at least 50%, 80%, 90%, 95%, 96%, 97%, 98%, 99% or 100% activity compared to MDM2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MDM2. In embodiments, MDM2 is the protein as identified by Accession No. Q00987, homolog or functional fragment thereof.

The terms “MDM4”, or “mouse double minute 4” as provided herein includes any of the recombinant or naturally-occurring forms of the mouse double minute 4 homolog (MDM4) or variants or homologs thereof that maintain MDM4 activity (e.g. within at least 50%, 80%, 90%, 95%, 96%, 97%, 98%, 99% or 100% activity compared to MDM4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MDM4. In embodiments, MDM4 is the protein as identified by Accession No. 015151, homolog or functional fragment thereof.

The term “extrachromosomal DNA” or “ecDNA” as used herein, refers to a deoxyribonucleotide polymer having a chromosomal composition (including histone proteins) that does not form part of a cellular chromosome. ecDNA molecules have a circular structure and are not linear, as compared to cellular chromosomes.

As used herein, the term “oncogene” refers to a gene capable of transforming a healthy cell into a cancer cell due to mutation or increased expression levels of said gene relative to a healthy cell. The terms “amplified oncogene” or “oncogene amplification” refer to an oncogene being present at multiple copy numbers (e.g., at least 2 or more) in a chromosome. Likewise, an “amplified extrachromosomal oncogene” is an oncogene, which is present at multiple copy numbers and the multiple copies of said oncogene form part of an extrachromosomal DNA molecule. In embodiments, the oncogene forms part of an extrachromosomal DNA. In embodiments, the amplified oncogene forms part of an extrachromosomal DNA. In embodiments, the extrachromosomal oncogene is EGFR. In embodiments, the extrachromosomal oncogene is c-Myc. In embodiments, the extrachromosomal oncogene is N-Myc. In embodiments, the extrachromosomal oncogene is cyclin D1. In embodiments, the extrachromosomal oncogene is ErbB2. In embodiments, the extrachromosomal oncogene is CDK4. In embodiments, the extrachromosomal oncogene is CDK6. In embodiments, the extrachromosomal oncogene is BRAF. In embodiments, the extrachromosomal oncogene is MDM2. In embodiments, the extrachromosomal oncogene is MDM4. In embodiments, the extrachromosomal oncogene is HRAS. In embodiments, the extrachromosomal oncogene is KRAS. In embodiments, the extrachromosomal oncogene is NRAS. In embodiments, the extrachromosomal oncogene is PDGFR. In embodiments, the extrachromosomal oncogene is VEGFR.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The terms “transcription start site” and “transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “transcription inhibitor” may be used herein to refer to molecules or substances that are able to directly or indirectly inhibit transcription. In embodiments, transcription inhibitors herein include, but are not limited to, 8-Cl-Ado, actinomycin D, AT8319M, cordycepin, dinaciclib, flavopiridol, fludarabine, P276-00, R547, RGB-286638, Roscovitine, or SNS-032.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

The term “promoter inhibitor” may be used herein to refer to molecules or substances that are able to inhibit transcription by interacting, directly or indirectly, with a promoter. In some embodiments, a promoter inhibitor comprises a transcriptional repressor.

The term “enhancer” as used herein refers to a region of DNA that may be bound by proteins (e.g., transcription factors) to increase the likelihood that transcription of a gene will occur. Enhancers may be about 50 to about 1500 base pairs in length Enhancers may be located downstream or upstream of the transcription initiation site that it regulates and may be several hundreds of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

The term “chromatin” as used herein refers to a complex of DNA and protein found in eukaryotic cells. Its primary function is packaging long DNA molecules into a more compact, denser shape, which prevents the strands from becoming tangled and plays important roles in reinforcing the DNA during cell division, preventing DNA damage, and regulating gene expression and DNA replication. The primary protein components of chromatin are histones, which bind to DNA and function as “anchors” around which the strands are wound. Regions of DNA containing genes which are actively transcribed are less tightly compacted and closely associated with RNA polymerases in a structure known as euchromatin, while regions containing inactive genes are generally more condensed and associated with structural proteins in heterochromatin. Chromatin compaction varies, as for instance during mitosis and meiosis, chromatin facilitates proper segregation of the chromosomes in anaphase.

The term “chromatin compaction” as used herein refers to the level of compactness or packaging of DNA molecules.

The term “chromatin accessibility” as used herein refers to the effect of chromatin structure modifications on gene transcription. Generally, the more accessible the chromatin is, the higher the level of transcriptional activity in the accessible region.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “aptamer” as used herein refers to an oligonucleotide or peptide molecule that binds to a specific target molecule.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present invention includes polypeptides that are substantially identical to any of SEQ ID NOs:1, 2, 3, 4, and 5.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=-4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

“Contacting” is used in accordance with its plain ordinary meaning and refers to the process of allowing at least two distinct species (e.g. nucleic acids and/or proteins) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two or more species to react, interact, or physically touch (e.g., bind), wherein the two or more species may be, for example, an extrachromosomal cancer-specific nucleic acid as described herein, aextrachromosomal cancer-specific nucleic acid binding RNA as described herein, and an endonuclease as described herein. In embodiments, contacting includes, for example, allowing an extrachromosomal cancer-specific nucleic acid, a cancer-specific nucleic acid binding RNA, and an endonuclease to contact one another to form an extrachromosomal nucleic acid peptide complex.

As used herein, the terms “binding,” “specific binding” or “specifically binds” refer to two or more molecules forming a complex (e.g., an extrachromosomal nucleic acid protein complex) that is relatively stable under physiologic conditions.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” or “standard control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma) , lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g. leukemia, lymphoma, B cell lymphoma, or multiple myeloma)) means that the disease (e.g. cancer, (e.g. leukemia, lymphoma, B cell lymphoma, or multiple myeloma)) is caused by (in whole or in part), or a symptom of the disease is caused by (in whole or in part) the substance or substance activity or function.

The term “prevent” refers to a decrease in the occurrence of disease symptoms in a patient. As indicated above, the prevention may be complete (no detectable symptoms) or partial, such that fewer symptoms are observed than would likely occur absent treatment.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid, antibody) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

An “epigenetic inhibitor” as used herein, refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Examples of HDAC inhibitors include Vorinostat, romidepsin, CI-994, Belinostat, Panobinostat , Givinostat, Entinostat, Mocetinostat, SRT501, CUDC-101, JNJ-26481585, or PCI24781. Examples of DNMT inhibitors include azacitidine and decitabine. Examples of HMT inhibitors include EPZ-5676. Examples of HDM inhibitors include pargyline and tranylcypromine. Examples of HAT inhibitors include CCT077791 and garcinol.

A “multi-kinase inhibitor” is a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Exemplary anti-cancer multi-kinase inhibitors include dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib.

“Selective” or “selectivity” or the like of a compound refers to the compound's ability to discriminate between molecular targets (e.g. a compound having selectivity toward HMT SUV39H1 and/or HMT G9a).

“Specific”, “specifically”, “specificity”, or the like of a compound refers to the compound's ability to cause a particular action, such as inhibition, to a particular molecular target with minimal or no action to other proteins in the cell (e.g. a compound having specificity towards HMT SUV39H1 and/or HMT G9a displays inhibition of the activity of those HMTs whereas the same compound displays little-to-no inhibition of other HMTs such as DOT1, EZH1, EZH2, GLP, MLL1, MLL2, MLL3, MLL4, NSD2, SET lb, SET7/9, SETS, SETMAR, SMYD2, SUV39H2).

As defined herein, the term “inhibition”, “inhibit”, “inhibiting” and the like in reference to a protein-inhibitor interaction means negatively affecting (e.g. decreasing) the activity or function of the protein or nucleic acid (e.g., amplified extrachromosomal oncogene or circular extrachromosomal DNA) relative to the activity or function of the protein or nucleic acid (e.g., amplified extrachromosomal oncogene or circular extrachromosomal DNA) in the absence of the inhibitor. In embodiments inhibition means negatively affecting (e.g. decreasing) the concentration or levels of a protein or nucleic acid (e.g., amplified extrachromosomal oncogene or circular extrachromosomal DNA) relative to the concentration or level of the protein or nucleic acid in the absence of the inhibitor. In embodiments, inhibition refers to reduction of a disease or symptoms of disease. In embodiments, inhibition refers to a reduction in the activity of a particular protein target or the level of a target nucleic acid (e.g., amplified extrachromosomal oncogene or circular extrachromosomal DNA). Thus, inhibition includes, at least in part, partially or totally blocking stimulation, decreasing, preventing, or delaying activation, or inactivating, desensitizing, or down-regulating signal transduction or enzymatic activity or the amount of a protein or nucleic acid (e.g., amplified extrachromosomal oncogene or circular extrachromosomal DNA). In embodiments, inhibition refers to a reduction of activity of a target protein resulting from a direct interaction (e.g. an inhibitor binds to the target protein). In embodiments, inhibition refers to a reduction of activity of a target protein or nucleic acid (e.g., amplified extrachromosomal oncogene or circular extrachromosomal DNA) from an indirect interaction (e.g. inhibitor binds to a protein that is involved in extrachromosomal oncogene amplification or circular extrachromosomal DNA replication, thereby preventing extrachromosomal oncogene amplification or circular extrachromosomal DNA replication).

The term “extrachromosomal DNA” or “ecDNA” as used herein, refers to a deoxyribonucleotide polymer of chromosomal composition (i.e. includes histone proteins) that does not form part of a cellular chromosome. ecDNA molecules have a circular structure and are not linear, as compared to cellular chromosomes. ecDNA may be found outside of the nucleus of a cell and may therefore also referred to as extranuclear DNA or cytoplasmic DNA. Circular extrachromosomal DNA (ecDNA) may be derived from genomic DNA, and may include repetitive sequences of DNA found in both coding and non-coding regions of chromosomes. The formation of ecDNA may occur independently of the cellular replication process. EcDNA may have a size from about 500,000 base pairs to about 5,000,000 base pairs.

An “ecDNA inhibitor” or “extrachromosomal DNA inhibitor” is an agent (e.g., a compound, small molecule, nucleic acid, protein) that negatively affects (e.g. decreases) the activity or function of ecDNA relative to the activity or function of ecDNA in the absence of the inhibitor. An ecDNA inhibitor as provided herein is a compound capable of reducing (decreasing) extrachromosomal oncogene amplification or circular extrachromosomal DNA replication relative to the absence of the inhibitor. In embodiments, the ecDNA inhibitor is a DNA repair pathway inhibitor.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition. In embodiments, the detectable agent is an HA tag. In embodiments, the HA tag includes the sequence set forth by SEQ ID NO:24. In embodiments, the HA tag is the sequence set forth by SEQ ID NO:24. In embodiments, the detectable agent is blue fluorescent protein (BFP). In embodiments, the BFP includes the sequence set forth by SEQ ID NO:30. In embodiments, the BFP is the sequence set forth by SEQ ID NO:30. In embodiments, the detectable agent is green fluorescent protein (GFP). In embodiments, the detectable agent is red fluorescent protein (RFP).

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The Applicants integrated ultrastructural imaging, long range-optical mapping, and computational analysis of whole genome sequencing to demonstrate unequivocally that ecDNA is circular. Pan-cancer analyses performed herein reveal that oncogenes encoded on ecDNA are among the most highly expressed genes in the transcriptome of tumors, linking elevated copy with very high levels of transcription. Quantitative assessment of the chromatin state, including ATAC-seq to measure the accessible genome and ATAC-see of cells in metaphase to examine open chromatin by spatial distribution, surprisingly reveal that while ecDNA is chromatinized, it lacks higher order compaction typical of chromosomes. In embodiments, ecDNA contains the most accessible DNA in the tumor genome. While not wishing to be held by theory, Applicants have found that, relative to linear chromosomal DNA, circular extrachromosomal DNA is distally active promoting expression across the ecDNA. Consequently, in embodiments, an inhibitor of a gene will have a surprisingly higher effect when directed to ecDNA than to the same gene on linear chromosomal DNA. Moreover, when the gene is an oncogene that resides on circular extrachromosomal DNA, in embodiments, a transcriptional inhibitor of that oncogene will have greater effect than on the same oncogene located on linear chromosomal DNA, and therefore, can be an effective cancer treatment.

### METHODS OF USE

In an aspect is provided a method of inhibiting expression of a first gene and a second gene in a subject, where the first gene and second gene are contained in a circular extrachromosomal DNA, the method including: administering an inhibitor of a promoter of the first gene to the subject, thereby inhibiting the expression of the first gene and the second gene. In embodiments, the first gene is an oncogene, the second gene is an oncogene, or both the first gene and second gene are oncogenes. In embodiments, the oncogene is KRAS, EGFR, c-Myc, N-Myc, cyclin D1, ErbB2, CDK4, CDK6, BRAF, MDM2, MDM4, ABL, AF4, HRX, AKT, ALK, ALK/NPM, AML1, AML1/MTG8, AXL, BCL-2, BCL-3, BCL-6, BCR/ABL, BRCA2, DBL, DEK/CAN, E2A, PBX1, PBX1/E2A, ENL/HRX, ERG/TLS, ERBBG, ERBB-2, ETS-1, EWS/FLI-1, FMS, FOS, FPS, GLI, GSP, HER2/neu, HOX11, HST, IL-3, INT-2, JUN, KIT, KS3, K-SAM, LBC, LCK, LMO1, LMO2, L-Myc, LYL-1, LYT-10, LYT-10/Cα1, MAS, MLL, MOS, MYB, BYH11/CBFB, NEU, OST, PAX-5, PIM-1, PRAD-1, RAF, RAR/PML, HRAS, NRAS, REL/NRG, RET, RHOM1, RHOM2, ROS, SKI, SIS, SET/CAN, SRC, TAL1, TAL2, TAN-1, TIAM1, TSC2, TRK, or a combination of two or more thereof. In embodiments, the oncogene is c-Myc, N-Myc, cyclin D1, CDK4, CDK6, MDM2, MDM4, ABL1, ABL2, AKT1, AKT2, ATF1, BCL11A, BCL2, BCL3, BCL6, BCR, BRCA2, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFR1OP, FRGR2, FUS, GOLGA5, GOPC, HMGA1, HMGA2, HRAS, IRF4, JUN, KIT, KRAS, LCK, LMO2, MAF, MAML2, MET, MITF, MLL, MPL, MYB, MYCL1, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SS18, TCL1A, TET2, TFG, TLX1, TPR, USP6, or a combination of two or more thereof. In embodiments, the oncogene is EGFR, c-Myc, N-Myc, cyclin D1, ErbB2, CDK4, CDK6, BRAF, MDM2, or MDM4. In embodiments, the oncogene is EGFR. In embodiments, the oncogene is c-Myc. In embodiments, the oncogene is N-Myc. In embodiments, the oncogene is cyclin D1. In embodiments, the oncogene is ErbB2. In embodiments, the oncogene is CDK4. In embodiments, the oncogene is CDK6. In embodiments, the oncogene is BRAF. In embodiments, the oncogene is MDM2. In embodiments, the oncogene is MDM4. In embodiments, the first gene and the second gene are the same. In embodiments, the first gene and the second gene are different. In embodiments, the first gene and the second gene are not within the same topologically associating domain (TAD). In embodiments, the promoter of the first gene interacts with an enhancer of the second gene contained in the circular extrachromosomal DNA. In embodiments, the inhibitor of the promoter of the first gene includes a transcriptional repressor domain. In embodiments, the transcriptional repressor domain is a Kruppel associated box (KRAB) domain. In embodiments, the method further includes inhibiting expression of a third gene contained in the circular extrachromosomal DNA with the inhibitor of a promoter of the first gene. In embodiments, the third gene is an oncogene. In embodiments, the oncogene is EGFR. In embodiments, the oncogene is c-Myc. In embodiments, the oncogene is N-Myc. In embodiments, the oncogene is cyclin D1. In embodiments, the oncogene is ErbB2. In embodiments, the oncogene is CDK4. In embodiments, the oncogene is CDK6. In embodiments, the oncogene is BRAF. In embodiments, the oncogene is MDM2. In embodiments, the oncogene is MDM4. In embodiments, the first gene, the second gene, and the third gene are the same or different. In embodiments, the first gene, the second gene, and the third gene are not within the same topologically associating domain (TAD). In embodiments, the promoter of the first gene interacts with an enhancer of the third gene contained in the circular extrachromosomal DNA. In embodiments, the method further includes inhibiting expression of all the genes contained in the circular extrachromosomal DNA with the inhibitor of a promoter of the first gene. In embodiments, the transcriptional inhibitor is an antisense nucleic acid. In embodiments, the transcriptional inhibitor is a siRNA. In embodiments, the transcriptional inhibitor is a microRNA. In embodiments, the transcriptional inhibitor is a ribonucleoprotein complex. In embodiments, the transcriptional inhibitor is a CRISPRi complex. In embodiments, the transcriptional inhibitor is a small molecule. In embodiments, transcription inhibitors herein include, but are not limited to, 8-Cl-Ado, actinomycin D, AT8319M, cordycepin, dinaciclib, flavopiridol, fludarabine, P276-00, R547, RGB-286638, Roscovitine, or SNS-032. In embodiments, the method includes determining whether the gene is contained in circular extrachromosomal DNA.

In an aspect is provided a method of inhibiting expression of a gene in a subject, wherein the gene is contained in circular extrachromosomal DNA, by administering a transcriptional inhibitor of the gene to the subject, thereby inhibiting the expression of the gene. In embodiments, the gene is an oncogene. Examples of oncogenes include, but are not limited to, EGFR, c-Myc, N-Myc, cyclin D1, ErbB2, CDK4, CDK6, BRAF, MDM2, or MDM4. In embodiments, the oncogene is EGFR. In embodiments, the oncogene is c-Myc. In embodiments, the oncogene is N-Myc. In embodiments, the oncogene is cyclin D1. In embodiments, the oncogene is ErbB2. In embodiments, the oncogene is CDK4. In embodiments, the oncogene is CDK6. In embodiments, the oncogene is BRAF. In embodiments, the oncogene is MDM2. In embodiments, the oncogene is MDM4. In embodiments, the transcriptional inhibitor is an antisense nucleic acid. In embodiments, the transcriptional inhibitor is a siRNA. In embodiments, the transcriptional inhibitor is a microRNA. In embodiments, the transcriptional inhibitor is a ribonucleoprotein complex. In embodiments, the transcriptional inhibitor is a CRISPRi complex. In embodiments, the transcriptional inhibitor is a small molecule. In embodiments, transcription inhibitors herein include, but are not limited to, 8-Cl-Ado, actinomycin D, AT8319M, cordycepin, dinaciclib, flavopiridol, fludarabine, P276-00, R547, RGB-286638, Roscovitine, or SNS-032. In embodiments, the method includes determining whether the gene is contained in circular extrachromosomal DNA.

In another aspect is provided a method of treating cancer in a subject in need thereof, where the cancer includes an oncogene on a circular extrachromosomal DNA, including: administering an inhibitor of a promoter of a first gene contained in the circular extrachromosomal DNA, where the inhibitor of the promoter of the first gene inhibits expression of the first gene and a second gene contained in the circular extrachromosomal DNA. In embodiments, the first gene is an oncogene, the second gene is an oncogene, or both the first gene and second gene are oncogenes. Examples of oncogenes include, but are not limited to, EGFR, c-Myc, N-Myc, cyclin D1, ErbB2, CDK4, CDK6, BRAF, MDM2, or MDM4. In embodiments, the oncogene is EGFR. In embodiments, the oncogene is c-Myc. In embodiments, the oncogene is N-Myc. In embodiments, the oncogene is cyclin D1. In embodiments, the oncogene is ErbB2. In embodiments, the oncogene is CDK4. In embodiments, the oncogene is CDK6. In embodiments, the oncogene is BRAF. In embodiments, the oncogene is MDM2. In embodiments, the oncogene is MDM4. In embodiments, the first gene and the second gene are the same or different. In embodiments, the first gene and the second gene are not within the same topologically associating domain (TAD). In embodiments, the promoter of the first gene interacts with an enhancer of the second gene contained in the circular extrachromosomal DNA. In embodiments, the inhibitor of the promoter of the first gene includes a transcriptional repressor domain. In embodiments, the transcriptional repressor domain is a Kruppel associated box (KRAB) domain. In embodiments, the method further includes administering a plurality of inhibitors of promoters of a plurality of genes contained in the circular extrachromosomal DNA. In embodiments, the promoter inhibitor is an antisense nucleic acid. In embodiments, the promoter inhibitor is a siRNA. In embodiments, the promoter inhibitor is a microRNA. In embodiments, the promoter inhibitor is a ribonucleoprotein complex. In embodiments, the promoter inhibitor is a CRISPRi complex. In embodiments, the promoter inhibitor is a small molecule. In embodiments, transcription inhibitors herein include, but are not limited to, 8-Cl-Ado, actinomycin D, AT8319M, cordycepin, dinaciclib, flavopiridol, fludarabine, P276-00, R547, RGB-286638, Roscovitine, or SNS-032. In embodiments, the method includes determining whether the gene is contained in circular extrachromosomal DNA.

In another aspect is provided a method of treating cancer in a subject in need thereof, where the cancer includes an oncogene on a circular extrachromosomal DNA, including administering a therapeutically effective amount of an agent that decreases chromatin accessibility of the circular extrachromosomal DNA, thereby treating the cancer in the subject. In embodiments, the agent increases chromatin compaction of the circular extrachromosomal DNA. In embodiments, the agent is a histone deacetylase inhibitor. In embodiments, the agent is a histone deacetylase. In embodiments, the method includes determining whether the gene is contained in circular extrachromosomal DNA.

In another aspect is provided a method of treating cancer in a human subject in need thereof, where cancer cells in the human subject include a first extrachromosomal oncogene forming part of a circular extrachromosomal DNA, the method including administering to the human subject a therapeutically effective amount of a transcriptional inhibitor of a first gene forming part of the circular extrachromosomal DNA in the cancer cells of the human subject, where the first gene and the first extrachromosomal oncogene do not form part of the same topologically associating domain (TAD). In embodiments, the method further includes a second extrachromosomal oncogene, where the first gene and the second extrachromosomal oncogene do not form part of the same topologically associating domain (TAD). In embodiments, the first gene is an oncogene. In embodiments, the first gene, the first extrachromosomal oncogene and the second extrachromosomal oncogene are independently different or the same. In embodiments, the first gene, the first extrachromosomal oncogene and the second extrachromosomal oncogene are independently EGFR, c-Myc, N-Myc, cyclin D1, ErbB2, CDK4, CDK6, BRAF, MDM2, or MDM4. In embodiments, the transcriptional inhibitor is a promoter inhibitor. In embodiments, the promoter inhibitor is an antisense nucleic acid, a siRNA, a microRNA, a CRISPRi complex or a small molecule. In embodiments, the promoter inhibitor includes a transcriptional repressor domain. In embodiments, the transcriptional repressor domain is a Kruppel associated box (KRAB) domain. In embodiments, the method further includes administering an effective amount of a plurality of transcriptional inhibitors. In embodiments, the transcriptional inhibitors are independently different. In embodiments, the method includes determining whether the gene is contained in circular extrachromosomal DNA.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized sepharose(TM), agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Compositions

In an aspect, provided herein are transcriptional inhibitors. In embodiments, provided herein are inhibitors of promoters or enhancers. In embodiments, the inhibitor decreases expression of circular extrachromosomal gene. In embodiments, the circular extrachromosomal gene is an oncogene. In embodiments, the transcriptional inhibitor is an antisense nucleic acid. In embodiments, the transcriptional inhibitor is a siRNA. In embodiments, the transcriptional inhibitor is a microRNA. In embodiments, the transcriptional inhibitor is a ribonucleoprotein complex. In embodiments, the transcriptional inhibitor is a CRISPRi complex. In embodiments, the transcriptional inhibitor is a small molecule. In embodiments, transcription inhibitors herein include, but are not limited to, 8-Cl-Ado, actinomycin D, AT8319M, cordycepin, dinaciclib, flavopiridol, fludarabine, P276-00, R547, RGB-286638, Roscovitine, or SNS-032. In embodiments, the method includes determining whether the gene is contained in circular extrachromosomal DNA. In embodiments, the promoter inhibitor is an antisense nucleic acid. In embodiments, the promoter inhibitor is a siRNA. In embodiments, the promoter inhibitor is a microRNA. In embodiments, the promoter inhibitor is a ribonucleoprotein complex. In embodiments, the promoter inhibitor is a CRISPRi complex.

In another aspect, an extrachromosomal nucleic acid protein complex is provided wherein the extrachromosomal nucleic acid protein complex includes an extrachromosomal cancer-specific nucleic acid bound to an endonuclease through a cancer-specific nucleic acid binding RNA.

The term “extrachromosomal cancer-specific nucleic acid” as used herein refers to a nucleic acid that forms part of an extrachromosomal DNA present in a cancer cell. The extrachromosomal cancer-specific nucleic acid may recombine with chromosomal DNA in a cancer cell and thereby become part of the cellular chromosome. The methods provided herein including embodiments thereof may detect extrachromosomal cancer-specific nucleic acids or amplified extrachromosomal oncogenes which originate from ecDNA, but during replication of the cancer cell become part of the cellular chromosome. In embodiments, the extrachromosomal cancer-specific nucleic acid is an oncogene. In embodiments, the extrachromosomal cancer-specific nucleic acid is an oncogene nucleic acid. In embodiments, the extrachromosomal cancer-specific nucleic acid is a non-essential gene nucleic acid. In embodiments, the extrachromosomal cancer-specific nucleic acid is an intragenic nucleic acid sequence. In embodiments, the extrachromosomal cancer-specific nucleic acid is a junction nucleic acid sequence. In embodiments, the extrachromosomal cancer-specific nucleic acid is amplified.

The term “cancer-specific nucleic acid binding RNA” refers to a polynucleotide sequence including the crRNA sequence and optionally the tracrRNA sequence. The crRNA sequence includes a guide sequence (i.e., “guide” or “spacer”) and a tracr mate sequence (i.e., direct repeat(s)). The term “guide sequence” refers to the sequence that specifies the target site (i.e., extrachromosomal cancer-specific nucleic acid).

In certain embodiments, the cancer-specific nucleic acid binding RNA is a single-stranded ribonucleic acid. In certain embodiments, the cancer-specific nucleic acid binding RNA is 10, 20, 30, 40, 50, 60, 70, 80, 90, 100 or more nucleic acid residues in length. In certain embodiments, the cancer-specific nucleic acid binding RNA is from 10 to 30 nucleic acid residues in length. In certain embodiments, the cancer-specific nucleic acid binding RNA is 20 nucleic acid residues in length. In certain embodiments, the length of the cancer-specific nucleic acid binding RNA can be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In certain embodiments, the cancer-specific nucleic acid binding RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In certain embodiments, the cancer-specific nucleic acid binding RNAis from 10 to 15, 10 to 20, 10 to 30, 10 to 40, or 10 to 50 residues in length.

In certain embodiments, the cancer-specific nucleic acid binding RNA has the sequence of SEQ ID NO:1. In certain embodiments, the cancer-specific nucleic acid binding RNA has the sequence of SEQ ID NO:2. In certain embodiments, the cancer-specific nucleic acid binding RNA has the sequence of SEQ ID NO:3. In certain embodiments, the cancer-specific nucleic acid binding RNA has the sequence of SEQ ID NO:4. In certain embodiments, the cancer-specific nucleic acid binding RNA has the sequence of SEQ ID NO:5. In certain embodiments, the cancer-specific nucleic acid binding RNA has the sequence of SEQ ID NO:6. In certain embodiments, the cancer-specific nucleic acid binding RNA has the sequence of SEQ ID NO:7. In certain embodiments, the cancer-specific nucleic acid binding RNA has the sequence of SEQ ID NO:8. In certain embodiments, the cancer-specific nucleic acid binding RNA has the sequence of SEQ ID NO:9. In certain embodiments, the cancer-specific nucleic acid binding RNA has sequence of SEQ ID NO:10. In certain embodiments, the cancer-specific nucleic acid binding RNA has the sequence of SEQ ID NO:11. In certain embodiments, the cancer-specific nucleic acid binding RNA has the sequence of SEQ ID NO:12. In certain embodiments, the cancer-specific nucleic acid binding RNA has the sequence of SEQ ID NO:13. In certain embodiments, the cancer-specific nucleic acid binding RNA has the sequence of SEQ ID NO:14. In certain embodiments, the cancer-specific nucleic acid binding RNA has the sequence of SEQ ID NO:15. In certain embodiments, the cancer-specific nucleic acid binding RNA has the sequence of SEQ ID NO:16. In certain embodiments, the cancer-specific nucleic acid binding RNA has the sequence of SEQ ID NO:17. In certain embodiments, the cancer-specific nucleic acid binding RNA has the sequence of SEQ ID NO:18.

The term “non-essential gene” as used herein refers to a gene of an extrachromosomal DNA that is not an oncogene and is located in close proximity to an oncogene. The non-essential gene may be amplified during oncogene amplification. Likewise, the term “intragenic sequence” as used herein refers to a nucleic acid sequence proximal to an oncogene. The intragenic sequence may be amplified during oncogene amplification. Amplification, as used herein, refers to the presence of multiple copies of a nucleic acid sequence.

The term “junction nucleic acid sequence” refers to a nucleic acid sequence that forms part of an extrachromosomal DNA and is formed upon the circularization of the extrachromosomal DNA. Inter- and intra-chromosomal rearrangements that occur during replication of a cancer cell within extrachromosomal DNA generate unique and novel nucleic acid junction sequences. The junction nucleic acid sequence may be targeted for the insertion of DNA double strand breaks in cancer cells since the junction nucleic acid sequences are specific for cancer cells and are not present in healthy cells.

In embodiments, the endonuclease is CRISPR associated protein 9 (Cas9), CxxC finger protein 1(Cpf1), or a Class II CRISPR endonuclease.

For specific proteins described herein (e.g., Cas9, Cpf1, and the like), the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

Thus, a “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 is an RNA-guided DNA endonuclease enzyme that binds a CRISPR (clustered regularly interspaced short palindromic repeats) nucleic acid sequence. In embodiments, the CRISPR nucleic acid sequence is a prokaryotic nucleic acid sequence. In embodiments, the Cas9 nuclease from Streptococcus pyogenes is targeted to genomic DNA by a synthetic guide RNA consisting of a 20-nt guide sequence and a scaffold. The guide sequence base-pairs with the DNA target, directly upstream of a requisite 5′-NGG protospacer adjacent motif (PAM), and Cas9 mediates a double-stranded break (DSB) about 3-base pair upstream of the PAM. In embodiments, the CRISPR nuclease from Streptococcus aureus is targeted to genomic DNA by a synthetic guide RNA consisting of a 21-23-nt guide sequence and a scaffold. The guide sequence base-pairs with the DNA target, directly upstream of a requisite 5′-NNGRRT protospacer adjacent motif (PAM), and Cas9 mediates a double-stranded break (DSB) about 3-base pair upstream of the PAM.

A “Cfp1” or “ Cfp1 protein” as referred to herein includes any of the recombinant or naturally-occurring forms of the Cfp1 (CxxC finger protein 1) endonuclease or variants or homologs thereof that maintain Cfp1 endonuclease enzyme activity (e.g. within at least 50%, 80%, 90%, 95%, 96%, 97%, 98%, 99% or 100% activity compared to Cfp1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cfp1protein. In embodiments, the Cfp1 protein is substantially identical to the protein identified by the UniProt reference number Q9P0U4 or a variant or homolog having substantial identity thereto.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) may generated in four sequential steps. First, two non-coding RNAs, the pre-crRNA array and tracrRNA, may be transcribed from the CRISPR locus. Second, tracrRNA may hybridize to the direct repeats of pre-crRNA, which is then processed into mature crRNAs containing individual spacer sequences. Third, the mature crRNA:tracrRNA complex may direct Cas9 to the DNA target consisting of the protospacer and the corresponding PAM via heteroduplex formation between the spacer region of the crRNA and the protospacer DNA. Finally, Cas9 may mediate cleavage of target DNA upstream of PAM to create a DSB within the protospacer.

In general, a guide sequence is any polynucleotide sequence having sufficient complementarity with a target polynucleotide sequence to hybridize with the target sequence (i.e., an extrachromosomal cancer-specific nucleic acid) and direct sequence-specific binding of a CRISPR complex to the target sequence (i.e., the extrachromosomal cancer-specific nucleic acid). In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any extrachromosomal cancer-specific nucleic acid. A guide sequence is designed to have complementarity with an extrachromosomal cancer-specific nucleic acid. Hybridization between the extrachromosomal cancer-specific nucleic acid and the guide sequence promotes the formation of a CRISPR complex. Full complementarity is not necessarily required, provided there is sufficient complementarity to cause hybridization and promote formation of a CRISPR complex. A guide sequence (spacer) may comprise any polynucleotide, such as DNA or RNA polynucleotides.

In general, a tracr mate sequence includes any sequence that has sufficient complementarity with a tracr sequence (i.e., a tracrRNA sequence) to promote one or more of: (1) excision of a guide sequence flanked by tracr mate sequences in a cell containing the corresponding tracr sequence; and (2) formation of a CRISPR complex at an extrachromosomal cancer-specific nucleic acid, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin.

Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence (i.e., the extrachromosomal cancer-specific nucleic acid), it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. Where the tracrRNA sequence is less than 100 (99 or less) nucleotides in length the sequence is one of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length.

In embodiments, the extrachromosomal cancer-specific nucleic acid binding RNA is at least in part complementary to the extrachromosomal cancer-specific nucleic acid.

In embodiments, the extrachromosomal nucleic acid protein complex forms part of a cell. In embodiments, the cell is a cancer cell. In embodiments, the cancer cell includes an extrachromosomal oncogene amplification.

In another aspect is provided an extrachromosomal nucleic acid protein complex including an extrachromosomal cancer-specific nucleic acid bound to an endonuclease through an extrachromosomal cancer-specific nucleic acid binding RNA. In embodiments, the extrachromosomal cancer-specific nucleic acid is an oncogene nucleic acid. In embodiments, the extrachromosomal cancer-specific nucleic acid is a non-essential gene nucleic acid. In embodiments, the extrachromosomal cancer-specific nucleic acid is an intragenic nucleic acid sequence. In embodiments, the extrachromosomal cancer-specific nucleic acid is a junction nucleic acid sequence. In embodiments, the extrachromosomal cancer-specific nucleic acid is an amplified extrachromosomal cancer-specific nucleic acid. In embodiments, the endonuclease is a CRISPR associated protein 9 (Cas9), a CxxC finger protein 1(Cpf1), or a Class II CRISPR endonuclease. In embodiments, the endonuclease is a TALEN. In embodiments, the endonuclease is a zinc finger. In embodiments, the endonuclease is a mega-nuclease. In embodiments, the extrachromosomal cancer-specific nucleic acid binding RNA is at least in part complementary to said extrachromosomal cancer-specific nucleic acid. In embodiments, the extrachromosomal nucleic acid protein complex forms part of a cell. In embodiments, the cell is a cancer cell. In embodiments, the cancer cell comprises an amplified extrachromosomal oncogene.

### EXAMPLES

### Example 1

### Materials and Methods

Cell culture. Human prostate cancer cell line PC3, colon cancer cell line COLO320DM and glioblastoma cell line U87 were purchased from ATCC and cultured in DMEM/F12 with 10% FBS. Human glioblastoma GBM39 tumor spheroid was derived from patient tissue, and cultured in DMEM/F12 with GlutaMAX, B27, 20 ng/ml EGF, 20 ng/ml FGF and 5 μg/ml heparin.

Metaphase chromosome spread. Cells in metaphase were obtained by KaryoMAX (Gibco) treatment at 0.1 μg/ml for 3 hr (PC3 and COLO320DM) or overnight (GBM39). Cells were washed with PBS and single cells were suspended in 75 mM KCl for 15-30 min Samples were then fixed by Carnoy's fixative (3:1 methanol:glacial acetic acid, v/v) and washed an additional three times with fixative before being dropped onto humidified glass coverslips.

FISH. Coverslips containing fixed cells in metaphase were aged overnight, briefly equilibrated by submerging in 2×SSC buffer, followed by dehydration in ascending ethanol series (70%, 85%, 100%) for 2 minutes each. Pre-warmed FISH probes (Empire Genomics) were added onto a slide, and the coverslip was applied and sealed with rubber cement. The FISH probe and sample were co-denatured on a 75° C. hotplate for 3 minutes, and hybridization was carried out overnight at 37° C. in a humidified chamber. The coverslips were removed and washed in 0.4×SSC at 72° C., followed by a final wash in 2×SSC/0.05% Tween-20, 2 minutes each. DNA was stained with DAPI (1 μg/mL; 2 minutes), washed with 2×SSC, mounting medium (VectaShield) was applied and the coverslip was mounted onto a glass slide.

Immunofluorescence on metaphase chromosome. Metaphase cells were obtained similarly by KaryoMAX treatment and KC1 swelling. Unfixed cells (2.5-4×10⁴) were spread onto a slide by Cytospin cytocentrifuge (Thermo Scientific). After aging overnight at 4° C., 100 μl primary and secondary antibodies in antibody diluent (DAKO) were applied sequentially onto the samples, with gentle washing by 2×SSC buffer with 0.1% Tween-20. Samples were then fixed by 4% paraformaldehyde in PBS, rinsed and mounted with ProLong Gold antifade mounting media with DAPI (Invitrogen).

Correlative light and electron microscopy. Fixed cells in metaphase were dropped onto Zeiss coverslips with fiducial markings. Images of DAPI-stained cells were captured with a Zeiss 880 Airyscan confocal microscope, and the locations of select cells were stored using the Shuttle and Find feature of the ZEN Black software. To correlate SEM with the DAPI-stained acquired images, the coverslip was briefly washed with ddH₂O and stained with 2% uranyl acetate for 2 minutes. The coverslip and holder were then loaded into the SEM and the same previously imaged DAPI-stained cells in metaphase were located using Shuttle and Find with ZEN Blue software. Images were captured using a Zeiss Sigma VP Scanning Electron Microscope and correlated with light microscope images.

Structured illumination microscopy. Cells in metaphase were prepared and dropped onto a glass coverslip. FISH was carried out as described, and images were captured with a GE (formerly Applied Precision) DeltaVision OMX V2 Structured Illumination microscope with a 100× Olympus PlanApo 1.4 NA objective and EMCCD 10 MHz camera mode. Structured Illumination reconstructions were performed using Softworx version 6.5.2, Weiner filter for 442 channel was set to 0.0060. Volume renderings were also done with Softworx version 6.5.2 software via the RGB Opacity method preset, and then these were used to generate 3D intensity plots of ecDNA.

Transmission electron microscopy. Cells in metaphase were dropped onto a glass coverslip and fixed in 2% glutaraldehyde/0.1 M cacodylate buffer. The sample was then stained in a 1% osmium tetroxide in 0.15M cacodylate buffer for 1 hour on ice, followed by 3 washes in 0.1 M cacodylate buffer for 15 minutes each. Cells were then immersed in 2% uranyl acetate in water for 1 hour on ice and dehydrated in a graded series of ethanol (20%, 50%, 70%, 90%, 100%) on ice for 15 minutes each. The sample was then embedded in Durcupan resin and polymerized overnight in a 60° C. oven, sectioned at 50-60 nm on a Leica UCT7 ultramicrotome, and picked up on a Formvar and carbon-coated copper grid. Sections were post-stained with 2% uranyl acetate for 5 minutes and Sato's lead stain for 1 minute. Images were captured at 25 kX using a Jeol 1400Plus TEM equipped with a 16 megapixel Gatan OneView camera.

Confocal microscopy. Immunofluorescence and ATAC-see images were acquired by Zeiss LSM880 Airyscan confocal microscope, using 63x Plan-APOChromat NA 1.4 oil lens. 20-30 Z-stacks (4.78 depth) were taken from each visual field, and Fast Airyscan processing was done by ZEN Black software in 3D mode at default settings [Wiener filter was 3.3, 3.9 and 4.2 for ATAC-see (Red), MYC FISH (Green), and DAPI (Blue), respectively]. Representative images were selected from the Z-stack with best brightness. The gain was 745, 785, and 700 for the Red, Green, and DAPI channels, respectively. The pinhole was automatically opened by the software for Fast Airyscan acquisition, and the pixel dwell time was 0.93 μs with no averaging. Double FISH images were captured with the Leica TCS SP8 confocal microscope. Image processing for highest resolution were obtained by using Leica Lightning Imaging Information Extraction Software. We used the proprietary Adaptive algorithm included in the Lightning software, including the following parameters: the pinhole was set to 0.5 Airy Units, with no cut-off, and 4 iterations were obtained per channel. The effective resolution achieved was 118 nm and was calculated using the half-width at half-max method, and measured from a single FISH signal.

Whole genome sequencing. Genomic DNA was extracted from cells using Qiagen kits. Sequencing libraries were prepared using TruSeq adapters (Illumina) and the KAPA HyperPlus kit, according to manufacturer's instructions (Kapa Biosystems). Briefly, 250 ng of DNA was used as input and enzyme-fragmented for 12 minutes to obtain mode fragment lengths of 350 bp. KAPA Pure Beads were used for double-sided size selection of 250-450 bp. DNA libraries were pooled and paired-end DNA sequencing (150 cycles) was performed on the NovaSeq S4.

Amplicon Architect. After the fastq files were aligned to the reference genome using bwa mem with default parameters, Amplicon Architect (AA) was run on the aligned reads using all regions with copy number greater than 5 as seeds. Default parameters were used as described in the documentation. Given mapped reads, AA automatically searches for other intervals participating in the amplicon, and then uses a carefully calibrated combination of Copy Number Variant (CNV) analysis and Structural Variant (SV) analysis. AA uses SV signatures (e.g. discordant paired-end reads and CNV boundaries) to partition all intervals into segments and build an amplicon graph. It assigns copy numbers to the segments by optimizing a balanced flow on the graph. As short reads do not span long repeated segments, they cannot disambiguate between multiple alternative structures. Therefore, high molecular weight DNA was used to generate optical mapping reads. The optical map reads were used to scaffold and disambiguate the graph, as described below.

Gene Classification. To predict putative ecDNA structures, a depth-first search algorithm was used to traverse the amplicon graph and identify cycles. Genes that lay on any cycle in the graph were designated as circular. Otherwise, they were designated as linear.

Isolation of high molecular weight (HMW) DNA for optical mapping. HMW DNA was extracted from GBM39 cells following manufacturer's instructions (BioNano Genomics #30026) with some modifications. The initial step in the procedure calls for the generation of agarose plugs containing the cell equivalent of ˜3 μg-9 μg of DNA (˜0.5-1.5 million diploid human cells), which is a critical step for recovering good quality HMW DNA. As GBM39 cells contain a roughly tetraploid amount of DNA with numerous extrachromosomal DNA¹, optimization of the DNA concentration was carried out as follows: Approximately 4.5 million GBM39 cells were spun down at 300 g for 10 minutes, washed twice with 0.5 mL cold Cell Buffer (BioNano #30026), and resuspended in 450 μL cold Cell Buffer. This solution was then split into three different tubes to approximate 9 μg of DNA (˜0.75 million cells), 6 μg of DNA (˜0.5 million cells), or 3 μg of DNA (˜0.25 million cells), spun down at 300 g for 5 minutes, and resuspended in Cell Buffer to reach a final volume of 66 μL. 40 μL of 2% agarose (BioRad CleanCut Agarose #170-3594) was added to the cells and incubated at 4° C. for 15 minutes to generate the agarose plugs. Within the plugs, the cells were lysed and digested with Proteinase K (Puregene #158920) and RNase A (Puregene #158922) per manufacturer's instructions. To stabilize, recover and clean the DNA, plugs were treated according to the manufacturer's instructions (BioNano Genomics #30026). Following dialysis, the DNA was homogenized and mechanically sheared by slowly pipetting the entire volume up and down with a non-filtered 200 μL tip until the sample reached an even consistency. The DNA was then equilibrated at room temperature for 3 days. Using a 2 μL aliquot, the DNA was diluted in Qubit BR buffer, sonicated for 10 minutes, and quantified using the Qubit dsDNA BR Assay kit (Invitrogen #Q32850). The sample obtained from the plug with ˜0.5 million cells yielded the best results with a mean DNA concentration of 61 ng/μL and a coefficient variation of 6.7% and was used for the nicking, labeling, repairing, staining (NLRS) reactions.

Optimization of the NLRS reactions and DNA loading onto IrysChip. The 2× nicking reaction (utilizing Nt.BspQI) and 1× labeling, repairing and staining reactions were performed as per manufacturer's instructions (BioNano Genomics #30024) using the recommended NEB reagents. Using a 2 μL aliquot, the DNA was sonicated for 20 minutes and the final DNA concentration was determined to be 3 ng/μL by Qubit dsDNA HS Assay kit (Life Technologies #Q32854). A total of 16 μL of nicked, labeled, repaired, and stained DNA was loaded onto the IrysChip (BioNano #FC-020-01) and run conditions were optimized on the Irys system to ensure efficient DNA loading onto the nanochannels using the Irys User Guide (BioNano Genomics #30047).

BioNano data analysis. 13 rounds of data (each round containing 30 cycles of data generation) were collected on the Irys platform to reach 0.791× reference coverage with molecules. Raw images were processed and long DNA molecules were detected and digitized by BioNano image-processing and analysis software AutoDetect³¹. Optical maps were generated by transforming the raw images into raw BNX files using the IrysView software system. The BNX files output from the BioNano instrument were then assembled into optical map contigs using the BioNano Irys assembly pipeline (v5122, default parameters). The segments discovered by Amplicon Architect were converted to an in-silico CMAP reference file and it was aligned to the assembled optical map contigs using AmpliconReconstructor. Alignment results were also confirmed using the BioNano RefAligner (v5122, default parameters). We produced a visualization of the resulting alignment using CycleViz.

RNA-seq. One microgram RNA extracted by RNeasy mini kit (QIAGEN) was prepared for sequencing with TruSeq RNA Library Prep Kit v2 (Illumina) according to the manufacturer's instruction. Briefly, after poly-A selection and fragmentation of the total RNA, first and second strand cDNA was synthesized and ligated with sequencing adapter. Products were then amplified for paired-end sequencing. Data were processed following the TCGA mRNA analysis pipeline. Expression level of mRNA was computed as fragments per kilobase of transcript per million mapped reads (FPKM) for cell line samples, or as upper quartile FPKM (FPKM-UQ) for both cell line and TCGA samples. Z score for FPKM-UQ was calculated as Z-score=(X-μ)/σ, where X is the FPKM-UQ of a given gene, μ and σ are the global mean and standard deviation of FPKM-UQ of a given sample's transcriptome respectively.

MNase-seq. One million cells were washed by calcium-free PBS and resuspended in 1 ml lysis buffer (10 mM pH 7.5 Tris-HCl, 10 mM NaCl, 3 mM MgCl₂, 0.5% IGEPAL CA-630, 0.15 mM spermine, 0.5 mM spermidine, with Roche EDTA-free complete protease inhibitor cocktail) on ice for 5 min. After centrifugation, cell pellets were resuspended in 160 μl digestion buffer (10 mM pH 7.5 Tris-HCl, 15 mM NaCl, 60 mM KCl, 0.15 mM spermine, 0.5 mM spermidine, with protease inhibitor cocktail) on ice. 0.004 Unit of micrococcal nuclease (NEB) in 40 μl digestion buffer (with 5 mM CaCl₂) was added to the suspension and incubated at room temperature for 10 min Digestion was halted by 200 μl stop buffer (20 mM EDTA, 20 mM EGTA, 1% SDS). DNA was then extracted, repaired by Fast DNA End Repair Kit (Thermo Scientific), adenylated by Klenow fragment (NEB), ligated with TruSeq adapters (Illumina) and amplified to make paired-end sequencing library.

ATAC-seq. Protocol was adapted from previous report³². Briefly, 100-500K cell nuclei were extracted by NPB buffer (5% BSA, 0.2% IGEPAL-CA630, 1 mM DTT, EDTA-free protease, in PBS) at 4° C. for 10 min. Tagmentation was done in TB buffer (33 mM Tris-acetate pH 7.8, 66 mM K-acetate, 11 mM Mg-acetate, 16% DMF) with Tn5 transposase (Illumina, San Diego, Calif.), at 37° C. for 30 min. DNA samples were then extracted and DNA libraries were generated by PCR. To compare ATAC-seq signal between circular and linear amplicons of TCGA samples, the normalized read counts were further normalized by segment length, DNA copy number, and the normalized read counts of the same length from a set of merged normal tissue controls.

ATAC-see. Protocol was adapted from the previously described publication²⁰ to apply on metaphase chromosome spreads. Briefly, metaphase sample was prepared as described onto a 1-mm coverslip and incubated with 50 nM of ATTO-590 transposome under 37° C. for 30 min in the dark. After washed twice by 2×SSC with 0.01% SDS for 15 min, and once by 2×SSC with 0.2% Tween-20 for 15 min, sample was subjected to FISH procedures, and finally stained by 1 μg/ml DAPI and mount with VECTASHIELD antifade mounting media (Vector Laboratories).

ATAC-see image analysis pipeline. A software tool called ECdetect' was further developed to analyze high resolution images and semantically segment DAPI-stained nuclei, chromosomes, and ecDNA. For each image, the ATAC-see intensity at each pixel location was captured by reading the pixel values. The pixel values were then grouped based on whether they belong to ecDNA, chromosomes, or nuclei, based on the semantic segmentation information from ECdetect. This was done by comparing the pixel locations of the ATAC-see intensities with the pixel locations of the segmentations.

PLAC-seq. Long-range chromatin interaction was probed by PLAC-seq as previous described^{22,23} using H3K27ac as the anchor (Diagenode C15200184-50), and applied MAPS pipeline³³ for the downstream data analysis. After removing PCR duplicates from the valid mapped reads, we kept all intra-chromosomal reads >1 Kb to quantify protein mediated long-range chromatin interactions, and all intra-chromosomal reads <=1 Kb on different strands to quantify ChIP enrichment level. Finally, we merged two replicates of the same cell type, resulting in ˜240 million and ˜218 million paired-end reads for GMB39 and U87 cells, respectively. To visualize chromatin interaction frequency at the EGFR locus, we first selected all paired-end reads within the ˜1.3 Mb region (chr7:54,830,975-56,117,062), and removed any reads overlapped with two deletion regions chr7:55,194,960-55,222,713, chr7:55,676,885-55,677,786) in GBM39 cells. Because this region is highly amplified as ecDNA in GBM39 cells, resulting much more reads, we downsampled reads in GBM39 sample to match the total number of reads at the same locus in U87 cells. Virtual 4C was generated at 10 Kb resolution.

4C-seq. Five million cells were cross-linked with 2% formaldehyde for 10 min at room temperature and quenched by 125 mM glycine for 5 min. Nuclei were isolated and digested with Csp6I (Thermo Scientific) overnight. Enzyme was inactivated by heating at 65° C. for 20 min and the digested chromatin was subjected for ligation by T4 ligase (Life Technologies) for 16 h. DNA was then purified with before the second digestion with DpnII (NEB) overnight. After enzyme inactivation, a second round of ligation was performed, and DNA was purified. 4.8 μg of DNA in total was used for PCR amplification. 4C-seq data was analyzed using 4C-ker³⁴. Reads were mapped to a reduced genome of unique 22 bp sequences flanking Csp6I sites in the hg19 genome.

CRISPR interference. Small guide RNAs (sgRNAs) targeting EGFR promoter within the 4C viewpoint were cloned into pLV-hU6-sgRNA-hUbC-dCas9-KRAB-T2a-Puro (Addgene plasmid #71236)³⁵ and lentivirus were produced by transfecting 293T cells (sgRNAs target sequences: SEQ ID NOs: 1, 2, and 3). GBM39 cells were then infected by lentivirus (MOI 3) for 4 days and subjected to RNA extraction and qPCR.

Immunoblotting. After transferring whole cell lysates to nitrocellulose membrane, the following antibodies were applied: Anti-EGFR at 1:5000 (EMD Millipore #06-847), anti-phospho-EGFR at 1:1000 (CST #3777S), anti-Tubulin at 1:2000 (CST #2125S), and secondary anti-rabbit IgG antibody at 1:2000 (CST #7074S).

Statistics. All sample size and statistical methods were indicated in the corresponding figure legends. If the data were normally distributed (by Shapiro-Wilk test) and homoscedastic (by Bartlett's test), Student's t-test (for two groups) and One-way ANOVA (>2 groups) were used to test the mean difference. Otherwise, Wilcoxon rank sum test (for two groups) and Kruskal-Wallis rank sum test (for >2 groups) were applied. For ATAC-seq long fragment size distribution data, Kolmogorov-Smirnov test (KS test) was used. For ATAC-see signal intensity data set, which have at least 3500 pixels sampled for ecDNA or chrDNA per image, Z-test was used to test the mean difference according to the central limit theorem. All statistical tests are two-sided. All boxplots are shown with median, upper and lower quartiles; whiskers indicate 1.5× interquartile range, and points as outliers.

### Example 2

### ecDNA is Organized into Topological Domains that Allow Transcriptional Activity of One Gene to Influence Transcription Activity of Another

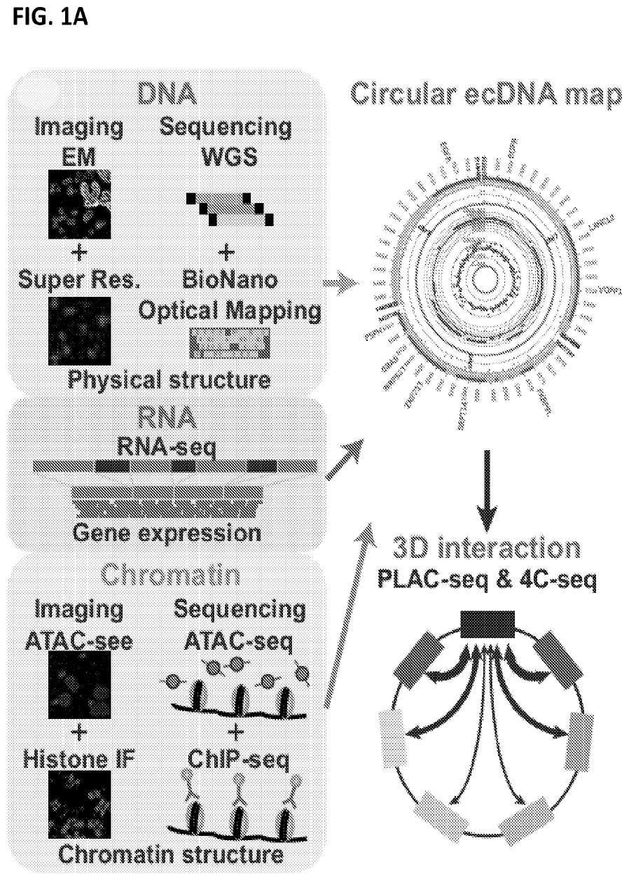

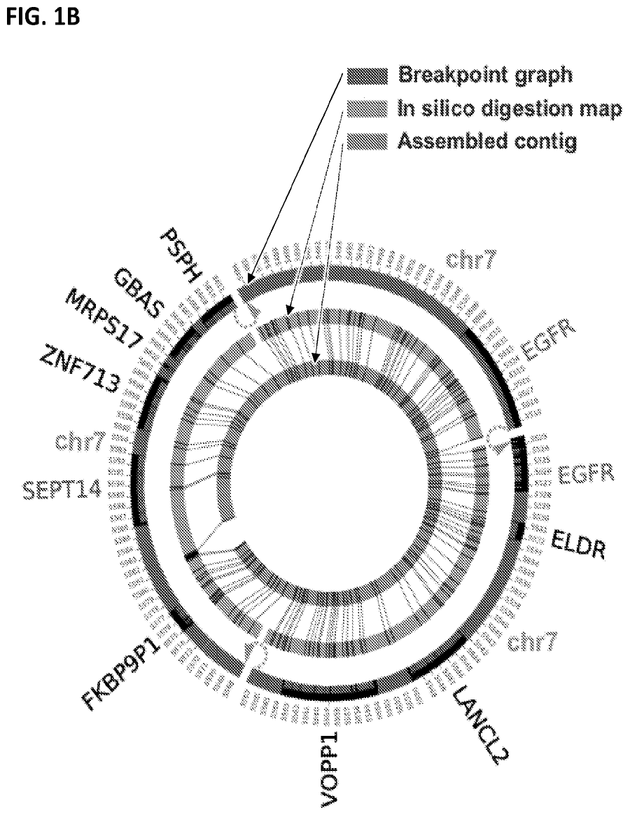

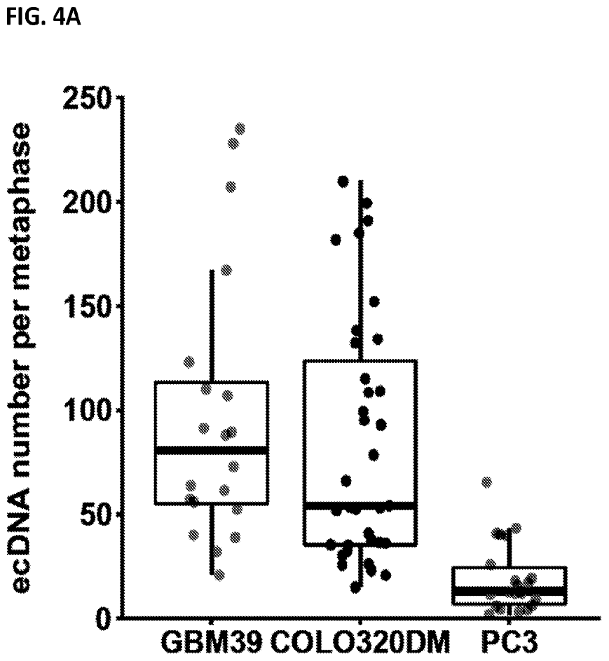

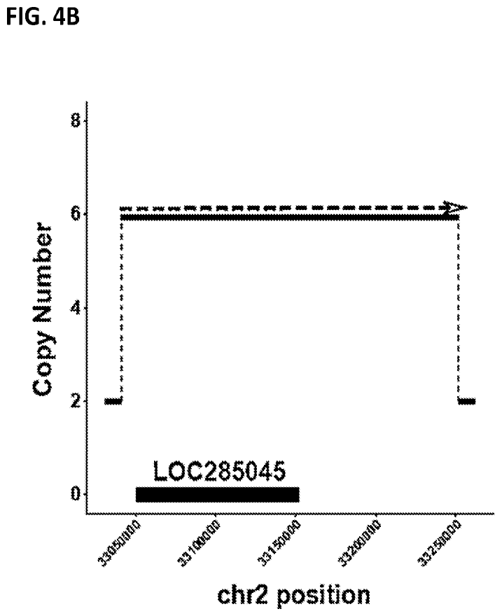

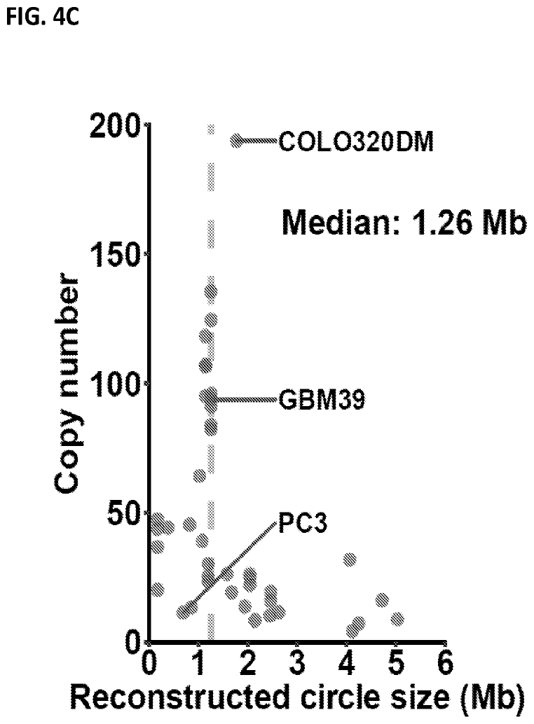

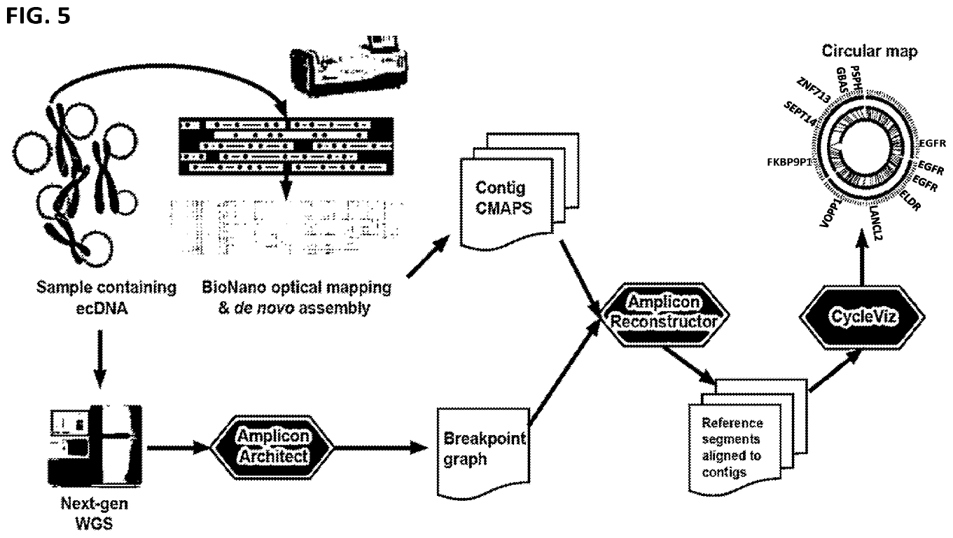

To understand ecDNA structure, transcription and chromatin organization, we studied three human cancer cell lines, GBM39, COLO320DM, AND PC3 (FIG. 4A) and clinical tumour samples from The Cancer Genome Atlas (TCGA), by integrating imaging and sequencing approaches (FIG. 1A). Previously, we used whole genome sequencing (WGS) to resolve ecDNA structure, deploying a computational tool, AmpliconArchitect (AA)^{1,9}, that classifies amplicons as circular or linear. Circular amplicons in GBM39 cells detected by this approach were confirmed to be extrachromosomal by fluorescence in situ hybridization (FISH) of tumor cells in metaphase. The reconstructed circular amplicon structure was supported by many paired-end discordant junctional reads and validated by Sanger sequencing. Genes detected on linear amplicons were found on chromosomal DNA (chrDNA; FIG. 4B). Reconstruction of 41 circular amplicons from 37 human cancer cell lines¹ revealed amplicon sizes ranged from 168 Kb to 5 Mb, with a median of 1.26 Mb (FIG. 4C), indicating that ecDNA size is heterogeneous.

AA infers a shape based on computational reconstruction of short, paired-end reads (100-200 bp), but does not unambiguously place large duplications in the structure. To augment our understanding of ecDNA shape based on its sequence, we integrated optical mapping of long-range reads (160,000 bp) of DNA, using the BioNano technology platform, which permits the development of a physical map based on long contiguous pieces of DNA^{10,11}. We developed a new tool, AmpliconReconstructor, to integrate the optical mapping contigs with AA based WGS-reconstructions, resolving a 1.3 MB circular, contiguous ecDNA molecule in GBM39 cells (FIG. 1B, and FIG. 5). Individual genes on the amplicon were visualized by super resolution (SR) confocal microscopy, showing that, for example, EGFR and SEPT14 can be on the same ecDNA, suggesting genes on ecDNA can be organized into topological domains.

To directly visualize ecDNA architecture, we captured images of COLO320DM cells containing MYC ecDNA, using SR 3D structured illumination microscopy (3D-SIM)¹², revealing circular ecDNA particles. To obtain more definitive evidence, we performed scanning and transmission electron microscopy (SEM and TEM). Correlative Light and Electron Microscopy analysis of COLO320DM cells, whose larger size ecDNA (FIG. 4C) was advantageous for visualization, demonstrated that DAPI (4′,6-diamidino-2-phenylindole) stained ecDNAs are circular. TEM analysis in GBM39 cells independently confirmed circular ecDNAs, including classical double minutes^{13,14}. Taken together, these results using DNA sequencing, optical mapping, super resolution 3D-SIM, SEM, and TEM demonstrate that these ecDNAs studied here are circular.

### Example 3

### The majority of Oncogene Transcriptional Activity Originates from ecDNA rather than Chromosomal DNA

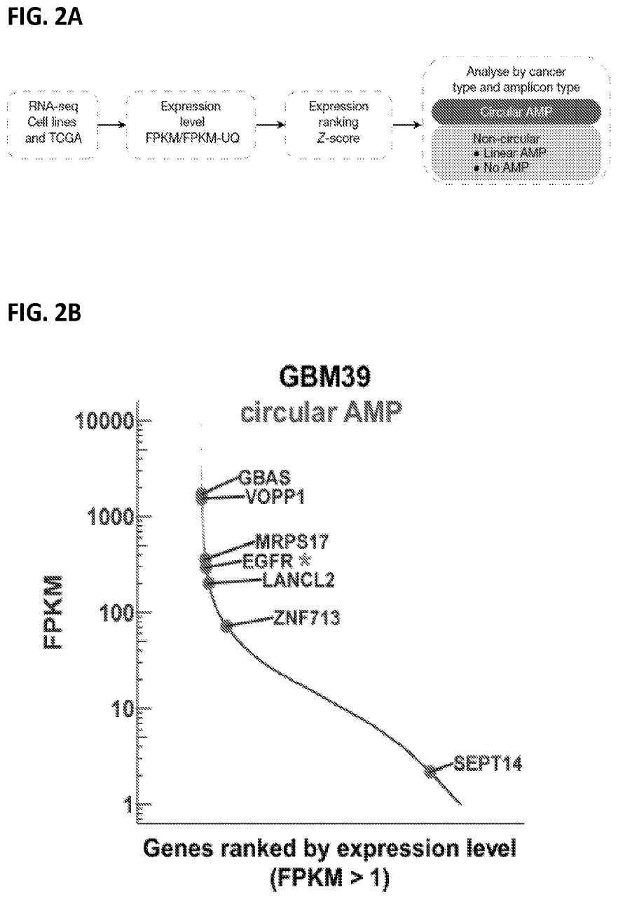

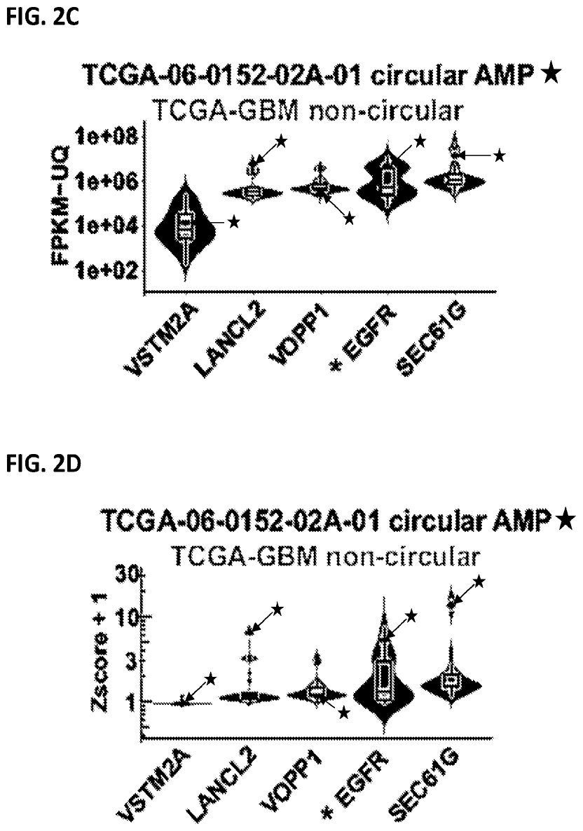

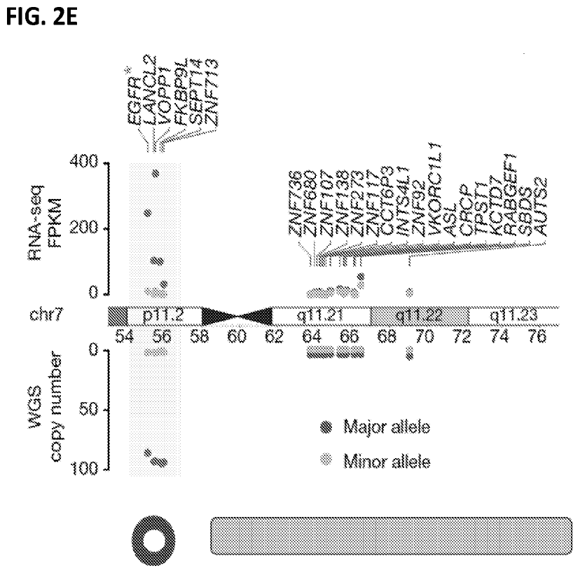

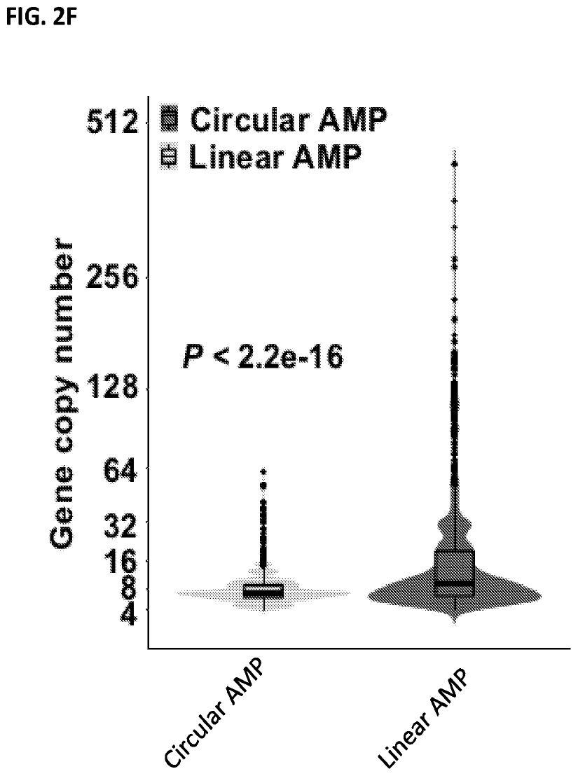

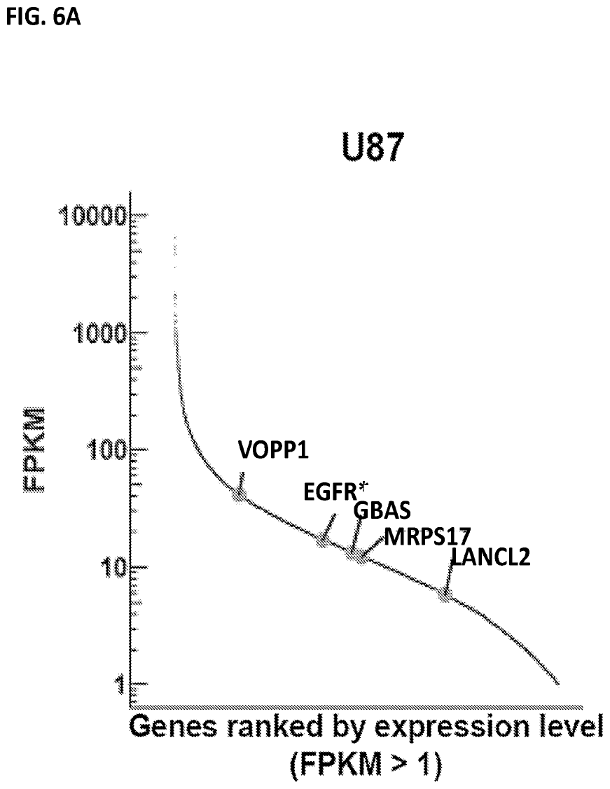

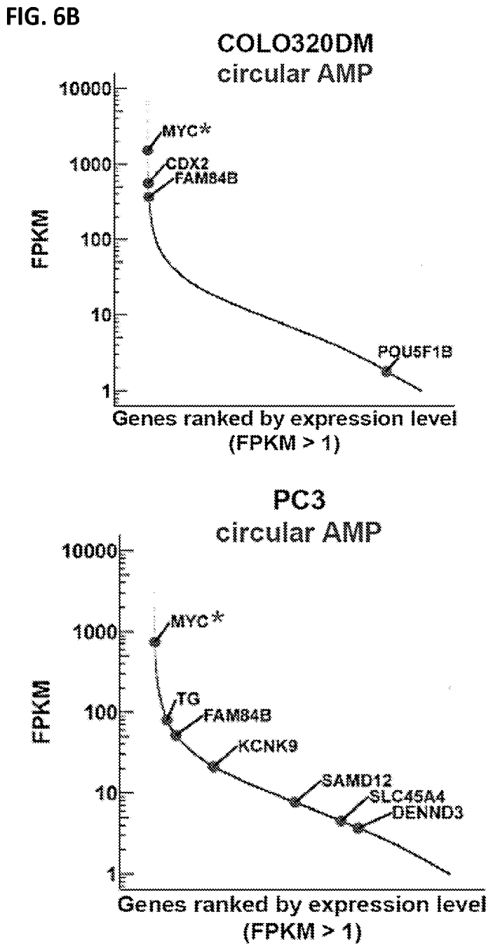

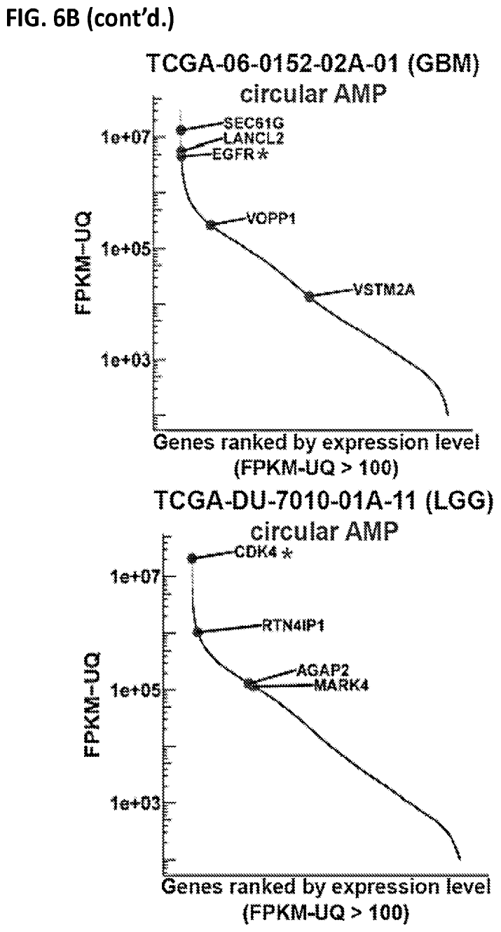

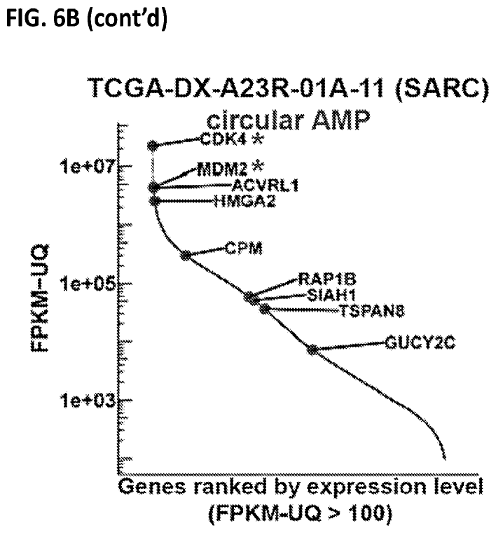

To determine the impact on transcription, we integrated RNA-seq with WGS from cancer cell lines and from TCGA clinical tumour samples of diverse histological types, revealing that genes encoded on ecDNA, particularly bona fide oncogenes, are among the most highly expressed genes in cancer genomes (FIGS. 2A, 2B, 6A, and 6B). Using our AA-based approach to determine if specific genes are amplified on circular ecDNA, we found that in cancer cell lines and clinical tumor samples, oncogenes amplified on ecDNA have significantly increased transcription compared to the same genes when they are not amplified by circularization (FIGS. 2C, 2D). We searched for single nucleotide polymorphisms in the WGS and RNA-seq data that permitted us to distinguish between transcription from genes on ecDNA and from their native chromosomal loci, revealing massively elevated transcription from genes encoded on ecDNAs (FIG. 2E). In fact, oncogenes encoded on ecDNA, including EGFR, MYC, CDK4 and MDM2, are among the top 1% of genes expressed in the cancer genomes (FIG. 2B).

The amount of RNA transcribed can be related to the amount of available DNA template. Oncogenes amplified on ecDNA were shown to achieve far higher copy number than the same genes amplified on linear structures (FIG. 2F). However, the amount of DNA template is not the only factor that determines gene transcription. Chromatin organization influences DNA's accessibility to the regulatory machinery of transcription^{4,16}. In some cases, oncogenes on ecDNA produced more transcripts, even when normalized to gene copy number. We initiated a deeper examination of other chromatin structural features that may contribute to the massively elevated expression of oncogenes amplified on ecDNA.

### Example 4

### ecDNA is More Transcriptionally Accessible than Chromosomal DNA

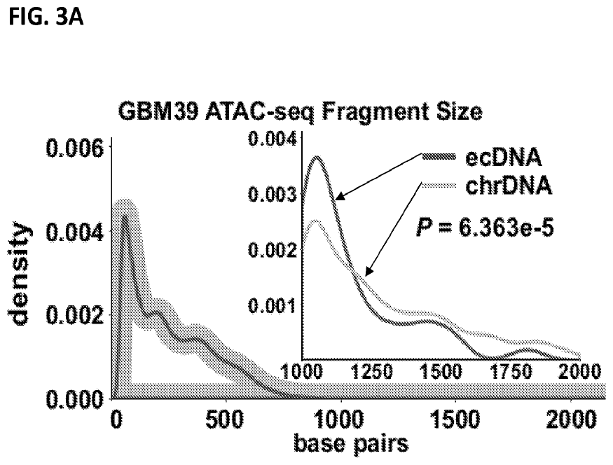

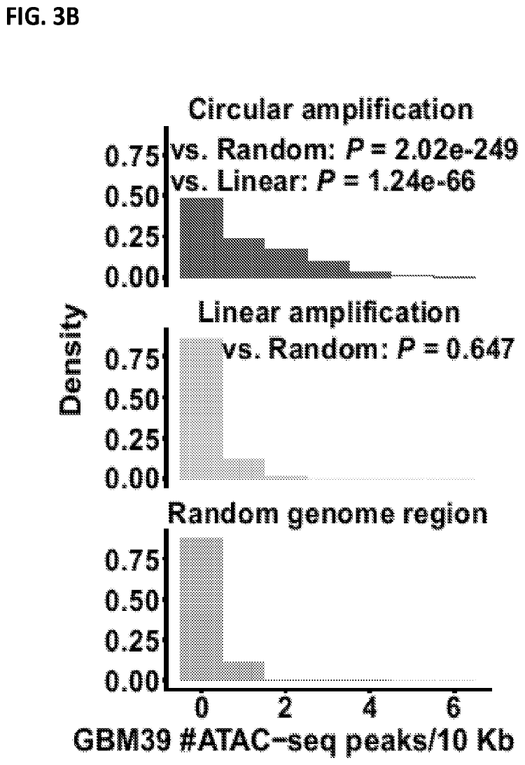

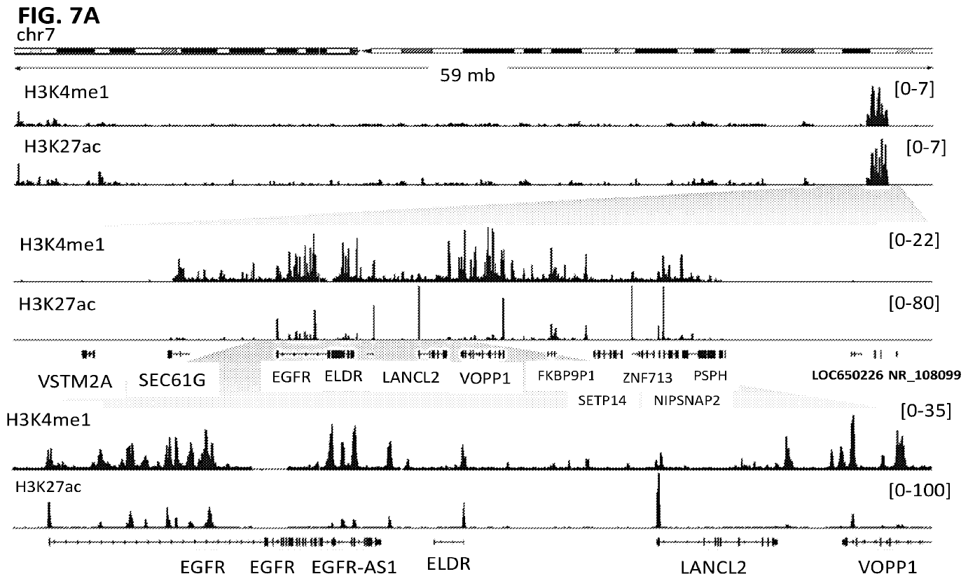

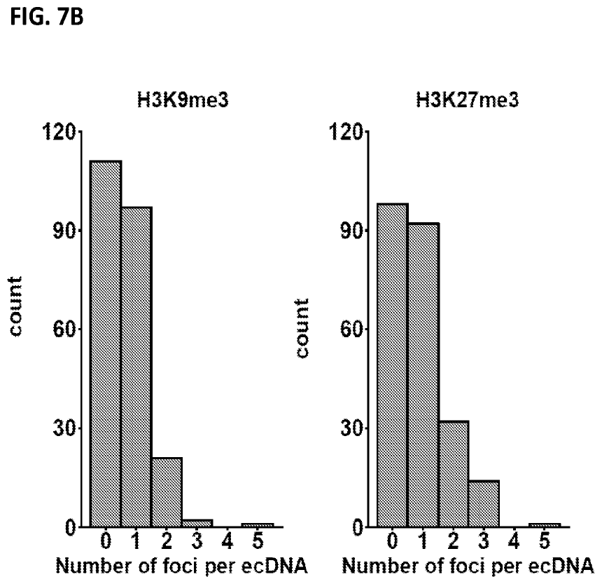

Most of the human genome is not transcribed in a given cell because it is tightly wound around histone octamers which in turn are packed into complex hierarchical structures, rendering the DNA inaccessible to transcription factors and the transcription machinery^{17,18.} We used complementary approaches to resolve the ecDNA chromatin landscape. First, we analyzed active and repressive histone marks by immunofluorescence analysis of cancer cells in metaphase and also performed H3K4me1/H3K27ac ChIP-seq analyses of actively cycling GBM39 cells, revealing the presence of active histone marks on ecDNA¹⁹ (FIG. 7A), and a concomitant paucity of repressive histone mark on GBM39 ecDNA (FIG. 7B). Second, we deployed the Assay for Transposase Accessible Chromatin using sequencing (ATAC-seq) and Micrococcal Nuclease digestion and sequencing (MNase-seq) to assess chromatin accessibility and to map nucleosome positions. Finally, we employed ATAC-see to directly visualize accessible chromatin²⁰. The periodic lengths distribution of DNA fragments generated by ATAC-seq and MNase-seq, demonstrated that ecDNA is chromatinized, comprised of nucleosome units (FIG. 3A). However, ecDNA displayed a significant deficit in the number of long fragments (>1200 bp) from ATAC-seq and MNase-seq, indicative of compacted nucleosomal arrays (FIG. 3A), and a significantly increased number of ATAC-seq peaks (FIG. 3B), indicating that the ecDNA chromatin landscape is more accessible than chrDNA, because its nucleosomal organization is less compacted.

The recent landmark study deciphering the chromatin accessibility landscape in primary cancer samples⁵ enabled us to examine chromatin accessibility in authentic clinical samples. By integrating ATAC-seq profiles with WGS data analyzed by AA, we found a significantly higher ATAC-seq signal in the DNA with predicted circular amplicons in clinical tumor samples, even after normalizing for DNA copy number. Even in isogenic cell lines, ecDNA is more accessible compared to the same locus amplified as homogeneous staining region (HSR)²¹ on chromosomes. Notably, the HSR region did not show a deficit in number of long ATAC-seq fragments as compared to ecDNA. We further validated that both the enhanced chromatin accessibility and active chromatin states are linked to the elevated transcription from the allele contained on highly amplified ecDNA.

We then applied the ATAC-see technology to analyze accessible chromatin in actively cycling cells in interphase by staining COLO320DM cells with ATAC-see and DAPI to label accessible chromatin and DNA, respectively, and to permit sorting of tumor cells in early G1 phase²⁰, followed by MYC-FISH to label ecDNAs. A striking positive correlation between ecDNA-containing MYC FISH signal and ATAC-see signal was seen, demonstrating highly accessible chromatin of ecDNA at single cell resolution. ecDNA remained similarly accessible during metaphase. Together, these data demonstrated that some of the most accessible chromatin in the genome of cancer cells resides on ecDNA, possibly due to the lower level of chromatin compaction. In fact, ATAC-see enabled us to identify unanticipated MYC ecDNAs in GBM39 cells because of their high signal, which was subsequently confirmed by ATAC-seq and WGS.

### Example 5

### ecDNA is Organized on Chromatin, but is Less Compact than Chromosomal DNA

To contextualize these genetic, transcriptional, and epigenetic features, we generated circular maps of ecDNA in cancer cell lines and primary tumour samples). These topologically informed maps highlighted the high DNA copy, high levels of transcription particularly of its constituent oncogenes, and high accessibility of its chromatin, bridging ecDNA circular structure with biological function. ecDNAs within a tumour can also vary in the size and composition (i.e. sequence), even when they contain the same oncogene. In GBM39 cells, the structure of EGFR-containing ecDNAs are uniform. Consequently, the WGS trace in its circular map is relatively uniform. In contrast, COLO320DM and PC3 cells contain diverse MYC-containing ecDNA populations, resulting in a more heterogenous WGS trace in the circular ecDNA plots.

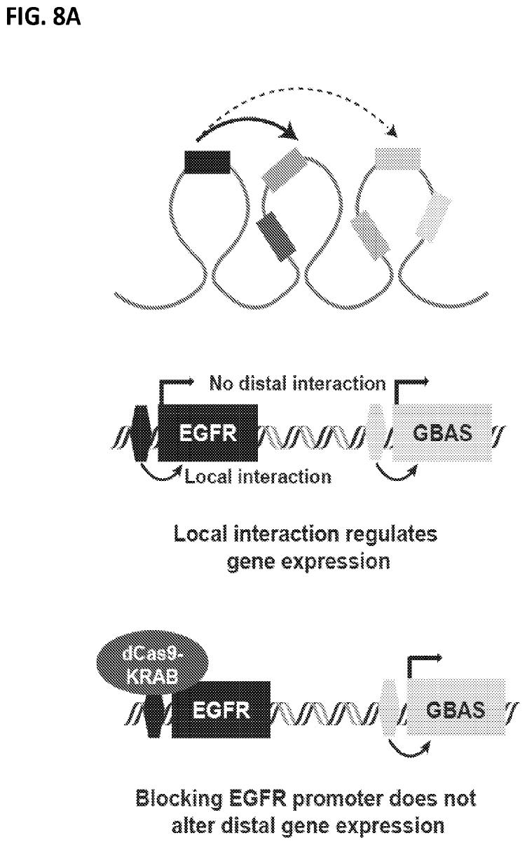

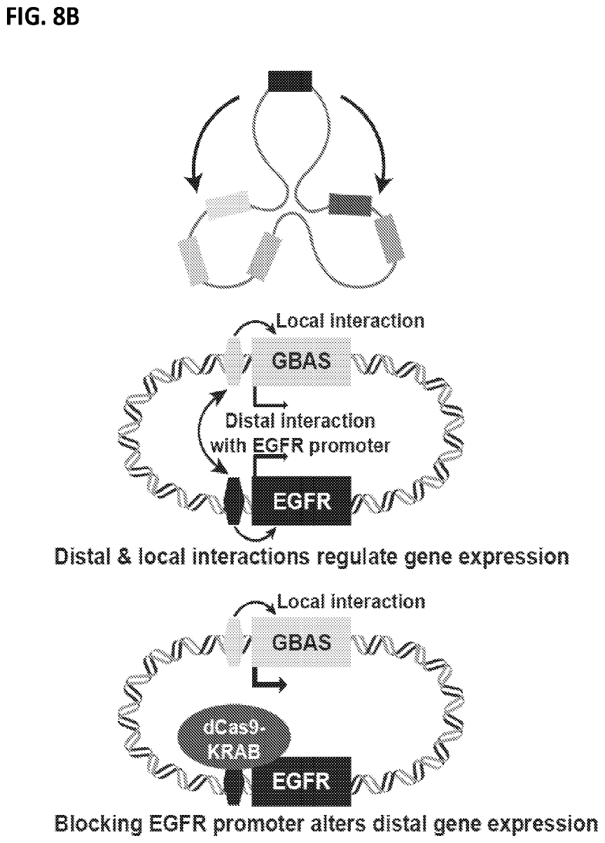

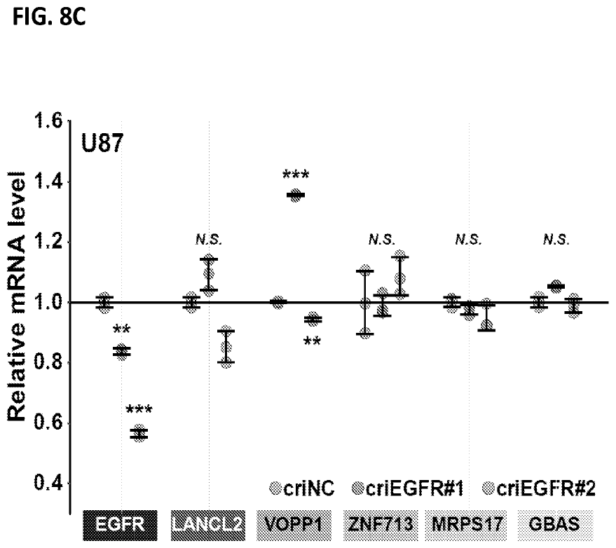

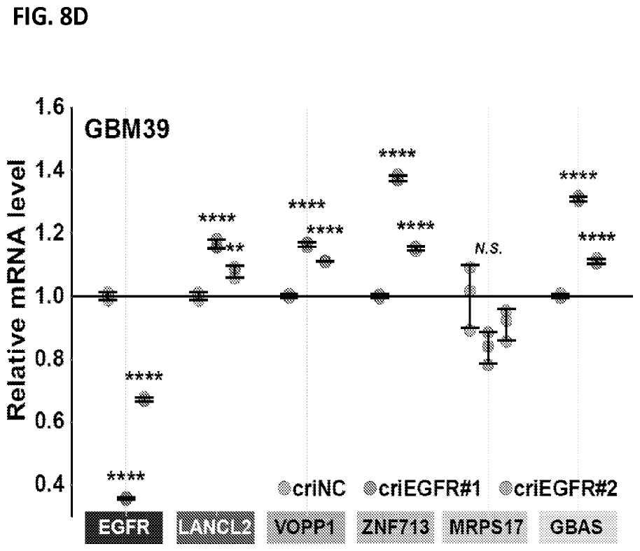

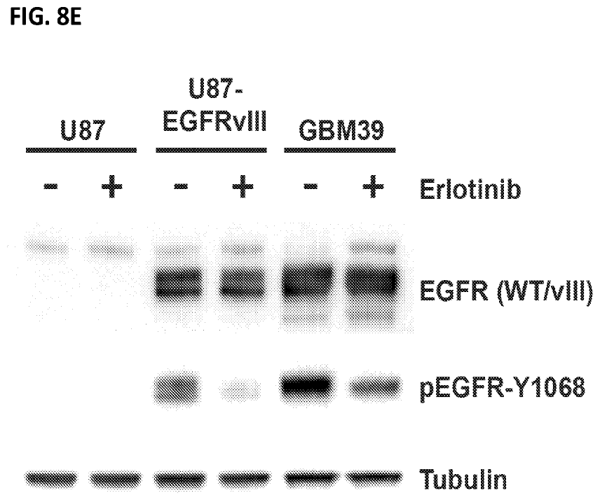

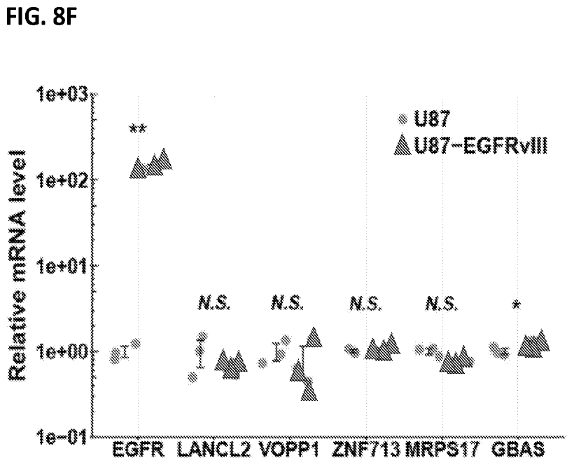

We performed Proximity Ligation-Assisted ChIP-seq²² (PLAC-seq, a.k.a. HiChIP²³) to map the chromatin 3D interactions genome-wide anchored at DNA bound by histone with H3K27ac modification in GBM39 cells. We also conducted Circular Chromosome Conformation Capture combined with high-throughput sequencing (4C-seq) to provide an independent assessment of chromatin contacts in GBM39 cells. Together with CTCF and cohesin subunit protein SMC3 ChIP-seq to examine the locations of factors important for chromatin domain organization²⁴, these data revealed: 1) the massive increase of diagonal corner reads on the heatmaps, and the rebound of the virtual 4C signal from ecDNA junction viewpoint, further provide orthogonal evidence to indicate that ecDNA is circular; 2)the binding of CTCF and cohesin demonstrate that ecDNA chromatin is well-organized, indicative of topologically associating domains; 3) downsampling the PLAC-seq/HiChIP reads from the GBM39 ecDNA region to a level comparable to the same region in U87 cells that lack ecDNA, still demonstrated notably increased distal interactions in active chromatin on ecDNA. Using the EGFR promoter as bait, the virtual 4C and actual 4C-seq independently demonstrated ultra-long-range chromatin contacts that can occur on ecDNA, which can effect distal gene expression, as indicated by CRISPR interference targeting catalytically inactive Cas9 (dCas9) fused to the Krüppel-associated box (KRAB) transcriptional repressor domain to mask the EGFR promoter (FIGS. 8A-F).

### Embodiments

Embodiment 1. A method of treating cancer in a subject in need thereof, wherein the cancer comprises an oncogene contained on a circular extrachromosomal DNA, comprising: administering a transcriptional inhibitor of a gene contained on the circular extrachromosomal DNA, wherein the transcriptional inhibitor of the gene inhibits expression of the oncogene contained in circular extrachromosomal DNA.

Embodiment 2. The method of embodiment 1, wherein the gene is the oncogene.

Embodiment 3. The method of embodiment 1, wherein the gene is not the oncogene.

Embodiment 4. The method of embodiments 1 or 3, wherein the gene and the oncogene are not contained on the same circular extrachromosomal DNA molecule.

Embodiment 5. The method of any one of embodiments 1 to 4, wherein the oncogene is MYC, cyclin D1, CDK4, CDK6, MDM2, MDM4, ABL1, ABL2, AKT1, AKT2, ATF1, BCL11A, BCL2, BCL3, BCL6, BCR, BRCA2, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFR1OP, FRGR2, FUS, GOLGA5, GOPC, HMGA1, HMGA2, HRAS, IRF4, JUN, KIT, KRAS, LCK, LMO2, MAF, MAML2, MET, MITF, MLL, MPL, MYB, MYCL1, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SS18, TCL1A, TET2, TFG, TLX1, TPR, or USP6.

Embodiment 6. The method of any of embodiments 1-4, wherein the oncogene is EGFR, c-Myc, N-Myc, cyclin D1, ErbB2, CDK4, CDK6, BRAF, MDM2, MDM4, KRAS, or C-MET.

Embodiment 7. The method of any of embodiments 1-6, wherein said transcriptional inhibitor comprises an antisense nucleic acid, a siRNA, a microRNA, a ribonucleoprotein complex, a CRISPRi complex, or a small molecule.

Embodiment 8. The method of embodiment 7, wherein the transcriptional inhibitor is a small molecule and the small molecule comprises 8-Cl-Ado, actinomycin D, AT8319M, cordycepin, dinaciclib, flavopiridol, fludarabine, P276-00, R547, RGB-286638, Roscovitine, or SNS-032

Embodiment 9. The method of any of embodiments 1-8, further comprising determining whether the oncogene and/or the gene are contained in circular extrachromosomal DNA.

Embodiment 10. The method of any of embodiments 1-9, wherein the cancer is a leukemia, a lymphoma, a melanoma, a neuroendocrine tumor, a carcinoma, or a sarcoma.

Embodiment 11. A method of treating cancer in a subject in need thereof, wherein the cancer comprises an oncogene contained on a circular extrachromosomal DNA, comprising:

administering a transcriptional inhibitor of a first gene contained on the circular extrachromosomal DNA, wherein the transcriptional inhibitor of the first gene inhibits expression of the first gene and the oncogene contained in circular extrachromosomal DNA.

Embodiment 12. The method of embodiment 11, wherein the first gene and the oncogene are on the same circular extrachromosomal DNA molecule.

Embodiment 13. The method of embodiment 11 or 12, wherein the first gene is an oncogene.

Embodiment 14. The method of embodiment 13, wherein the oncogene is EGFR, c-Myc, N-Myc, cyclin D1, ErbB2, CDK4, CDK6, BRAF, MDM2, or MDM4.

Embodiment 15. The method of embodiment 11, wherein the first gene and the oncogene are the same gene.

Embodiment 16 . The method of embodiment 11, wherein the first gene and the oncogene are the different genes.

Embodiment 17. The method of any one of embodiments 11-16, wherein the first gene and the oncogene are not within the same topologically associating domain (TAD).

Embodiment 18. The method of any one of embodiments 11-17, wherein the first gene comprises a promoter, the second gene comprises an enhancer, and the promoter of the first gene interacts with an enhancer of the second gene contained on the circular extrachromosomal DNA.

Embodiment 19. The method of any one of embodiments 11-18, wherein said transcriptional inhibitor comprises an antisense nucleic acid, a siRNA, a microRNA, a ribonucleoprotein complex, a CRISPRi complex, or a small molecule.

Embodiment 20. The method of embodiment 19, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

Embodiment 21. The method of any one of embodiments 11-18, wherein the transcriptional inhibitor of the first gene comprises a transcriptional repressor domain.

Embodiment 22. The method of embodiment 19, wherein the transcriptional repressor domain is a Kruppel associated box (KRAB) domain.

Embodiment 23. The method of embodiment 11, further comprising administering a plurality of transcriptional inhibitors of a plurality of genes contained on the circular extrachromosomal DNA.

Embodiment 24. The method of any one of embodiments 11-23, further comprising determining whether the first gene and/or the second gene are contained on circular extrachromosomal DNA.

Embodiment 25. The method of any one of embodiments 11-24, wherein the cancer is a leukemia, a lymphoma, a melanoma, a neuroendocrine tumor, a carcinoma, or a sarcoma.

Embodiment 26. A method of treating cancer in a subject in need thereof, wherein the cancer comprises an oncogene on a circular extrachromosomal DNA, comprising administering a therapeutically effective amount of an agent that decreases chromatin accessibility of the circular extrachromosomal DNA, thereby treating the cancer in the subject.

Embodiment 27. The method of embodiment 13, wherein the agent increases chromatin compaction of the circular extrachromosomal DNA.

Embodiment 28. The method of any one of embodiments 26-27, wherein said agent is an antisense nucleic acid, a siRNA, a microRNA, a ribonucleoprotein complex, a CRISPRi complex, or a small molecule.

Embodiment 29. The method of embodiment 28, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

Embodiment 30. The method of any one of embodiments 26-28, further comprising determining whether the oncogene is contained on circular extrachromosomal DNA.

Embodiment 31. The method of any one of embodiments 26-30, wherein the cancer is a leukemia, a lymphoma, a melanoma, a neuroendocrine tumor, a carcinoma, or a sarcoma.

Embodiment 32. A method of treating cancer in a subject in need thereof, wherein cancer cells in the subject comprise a first extrachromosomal oncogene forming part of a circular extrachromosomal DNA, the method comprising administering to said subject a therapeutically effective amount of a transcriptional inhibitor of a first gene forming part of the circular extrachromosomal DNA in the cancer cells of the subject, wherein the first gene and the first extrachromosomal oncogene do or do not form part of a same topologically associating domain (TAD).

Embodiment 33. The method of embodiment 32, further comprising inhibiting expression of a second extrachromosomal oncogene, wherein the first gene and the second extrachromosomal oncogene do not form part of the same topologically associating domain (TAD).

Embodiment 34. The method of embodiment 32 or 33, wherein the first gene is an oncogene.

Embodiment 35. The method of embodiment 34, wherein the first gene, the first extrachromosomal oncogene and the second extrachromosomal oncogene are independently different.

Embodiment 36 . The method of embodiment 34, wherein the first gene, the first extrachromosomal oncogene and the second extrachromosomal oncogene are the same.

Embodiment 37. The method of embodiment 35, wherein said first gene, said first extrachromosomal oncogene and said second extrachromosomal oncogene are independently KRAS, C-MET, EGFR, c-Myc, N-Myc, cyclin D1, ErbB2, CDK4, CDK6, BRAF, MDM2, or MDM4.

Embodiment 38. The method of any one of embodiments 32-37, wherein said transcriptional inhibitor is a promoter inhibitor.

Embodiment 39. The method of embodiment 38, wherein said promoter inhibitor comprises an antisense nucleic acid, a siRNA, a microRNA, a CRISPRi complex, a ribonucleoprotein complex, or a small molecule.

Embodiment 40. The method of embodiment 39, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

Embodiment 41. The method of embodiment 38, wherein said promoter inhibitor comprises a transcriptional repressor domain.

Embodiment 42. The method of embodiment 41, wherein said transcriptional repressor domain is a Kruppel associated box (KRAB) domain.

Embodiment 43. The method of any one of embodiments 32-42, comprising administering an effective amount of a plurality of transcriptional inhibitors.

Embodiment 44. The method of embodiment 43, wherein said transcriptional inhibitors are independently different.

Embodiment 45 . The method of any one of embodiments 32-44, further comprising determining whether said first extrachromosomal oncogene is contained on circular extrachromosomal DNA.

Embodiment 46. The method of any one of embodiments 32-45, wherein the cancer is a leukemia, a lymphoma, a melanoma, a neuroendocrine tumor, a carcinoma, or a sarcoma.

Embodiment 47. A method of inhibiting expression of a gene in a subject, wherein the gene is contained on circular extrachromosomal DNA, comprising:

administering a transcriptional inhibitor of the gene to the subject, thereby inhibiting the expression of the gene.

Embodiment 48. The method of embodiment 47, wherein the gene is an oncogene.

Embodiment 49. The method of embodiment 48, wherein the oncogene is MYC, cyclin D1, CDK4, CDK6, MDM2, MDM4, ABL1, ABL2, AKT1, AKT2, ATF1, BCL11A, BCL2, BCL3, BCL6, BCR, BRCA2, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFR1OP, FRGR2, FUS, GOLGA5, GOPC, HMGA1, HMGA2, HRAS, IRF4, JUN, KIT, KRAS, LCK, LMO2, MAF, MAML2, MET, MITF, MLL, MPL, MYB, MYCL1, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SS18, TCL1A, TET2, TFG, TLX1, TPR, or USP6.

Embodiment 50. The method of embodiment 48, wherein the oncogene is KRAS, C-MET, EGFR, c-Myc, N-Myc, cyclin D1, ErbB2, CDK4, CDK6, BRAF, MDM2, or MDM4.

Embodiment 51. The method of any of embodiments 47-50, wherein the transcriptional inhibitor comprises an antisense nucleic acid, a siRNA, a microRNA, a ribonucleoprotein complex, a CRISPRi complex, or a small molecule.

Embodiment 52. The method of embodiment 51, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

Embodiment 53. The method of any of embodiments 47-51, further comprising determining whether the gene is contained on circular extrachromosomal DNA.

Embodiment 54. A method of inhibiting expression of a first gene and a second gene in a subject, wherein the first gene and second gene are contained on circular extrachromosomal DNA, comprising:

administering a transcriptional inhibitor of the first gene to the subject, thereby inhibiting the expression of the first gene and the second gene.

Embodiment 55. The method of embodiment 54, wherein the first gene and the second gene are on the same circular extrachromosomal DNA molecule.

Embodiment 56. The method of embodiment 54 or 55, wherein the first gene is an oncogene, the second gene is an oncogene, or both the first gene and second gene are oncogenes.

Embodiment 57. The method of embodiment 56, wherein the oncogene is KRAS, C-MET, EGFR, c-Myc, N-Myc, cyclin D1, ErbB2, CDK4, CDK6, BRAF, MDM2, or MDM4.

Embodiment 58. The method of embodiment 54, wherein the first gene and the second gene are the same.

Embodiment 59. The method of embodiment 54, wherein the first gene and the second gene are different.

Embodiment 60. The method of any one of embodiments 54-58, wherein the first gene and the second gene are not within the same topologically associating domain (TAD).

Embodiment 61. The method of any one of embodiments 54-60, wherein the first gene comprises a promoter, the second gene comprises an enhancer, and the promoter of the first gene interacts with the enhancer of the second gene contained on the circular extrachromosomal DNA.

Embodiment 62. The method of any one of embodiments 54-61, wherein said transcriptional inhibitor comprises an antisense nucleic acid, a siRNA, a microRNA, a ribonucleoprotein complex, a CRISPRi complex, or a small molecule.

Embodiment 63. The method of embodiment 62, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

Embodiment 64. The method of any one of embodiments 54-62, wherein the transcriptional inhibitor of the first gene comprises a transcriptional repressor domain.

Embodiment 65. The method of embodiment 64, wherein the transcriptional repressor domain is a Kruppel associated box (KRAB) domain.

Embodiment 66. The method of any one of embodiments 54-65, further comprising inhibiting expression of a third gene contained on the circular extrachromosomal DNA with the transcriptional inhibitor of the first gene.

Embodiment 67. The method of embodiment 66, wherein the third gene is an oncogene.

Embodiment 68. The method of embodiment 67, wherein the oncogene is KRAS, C-MET, EGFR, c-Myc, N-Myc, cyclin D1, ErbB2, CDK4, CDK6, BRAF, MDM2, or MDM4.

Embodiment 69. The method of embodiment 54, wherein the first gene, the second gene, and the third gene are the same.

Embodiment 70. The method of embodiment 54, wherein the first gene, the second gene, and the third gene are different.

Embodiment 71. The method of any one of embodiments 54-69, wherein the first gene and the third gene are not within the same topologically associating domain (TAD).

Embodiment 72. The method of any one of embodiments 54-71, wherein the first gene comprises a promoter, the third gene comprises an enhancer, and the promoter of the first gene interacts with the enhancer of the third gene contained on the circular extrachromosomal DNA.

Embodiment 73. The method of any one of embodiments 54-72, further comprising inhibiting expression of all the genes contained on the circular extrachromosomal DNA with the inhibitor of a promoter of the first gene.

Embodiment 74. The method of any one of embodiments 54-73, further comprising determining whether the first gene and/or the second gene are contained on the circular extrachromosomal DNA.

Embodiment 75. A transcriptional inhibitor of a gene contained in a circular extrachromosomal DNA for use in any of the methods of embodiments 1-74.

### REFERENCES

1. Turner, K. M. et al. Extrachromosomal oncogene amplification drives tumour evolution and genetic heterogeneity. Nature 543, 122-125, doi:10.1038/nature21356 (2017).

2. Verhaak, R. G. W., Bafna, V. & Mischel, P. S. Extrachromosomal oncogene amplification in tumour pathogenesis and evolution. Nature reviews. Cancer 19, 283-288, doi:10.1038/s41568-019-0128-6 (2019).

3. Gibcus, J. H. & Dekker, J. The hierarchy of the 3D genome. Mol Cell 49, 773-782, doi:10.1016/j.molce1.2013.02.011 (2013).

4. Dixon, J. R., Gorkin, D. U. & Ren, B. Chromatin Domains: The Unit of Chromosome Organization. Mol Cell 62, 668-680, doi:10.1016/j.molce1.2016.05.018 (2016).

5. Corces, M. R. et al. The chromatin accessibility landscape of primary human cancers. Science 362, doi:10.1126/science.aav1898 (2018).

6. Hnisz, D. et al. Activation of proto-oncogenes by disruption of chromosome neighborhoods. Science 351, 1454-1458, doi:10.1126/science.aad9024 (2016).

7. Moller, H. D. et al. Circular DNA elements of chromosomal origin are common in healthy human somatic tissue. Nat Commun 9, 1069, doi:10.1038/s41467-018-03369-8 (2018).

8. Shibata, Y. et al. Extrachromosomal microDNAs and chromosomal microdeletions in normal tissues. Science 336, 82-86, doi:10.1126/science.1213307 (2012).

9. Deshpande, V. et al. Exploring the landscape of focal amplifications in cancer using AmpliconArchitect. Nat Commun 10, 392, doi:10.1038/s41467-018-08200-y (2019).

10. Mendelowitz, L. & Pop, M. Computational methods for optical mapping. Gigascience 3, 33, doi:10.1186/2047-217X-3-33 (2014).

11. Mak, A. C. et al. Genome-Wide Structural Variation Detection by Genome Mapping on Nanochannel Arrays. Genetics 202, 351-362, doi:10.1534/genetics.115.183483 (2016).

12. Demmerle, J. et al. Strategic and practical guidelines for successful structured illumination microscopy. Nat Protoc 12, 988-1010, doi:10.1038/nprot.2017.019 (2017).

13. Schimke, R. T. Gene amplification in cultured animal cells. Cell 37, 705-713, doi:10.1016/0092-8674(84)90406-9 (1984).

14. Storlazzi, C. T. et al. Gene amplification as double minutes or homogeneously staining regions in solid tumors: origin and structure. Genome Res 20, 1198-1206, doi:10.1101/gr.106252.110 (2010).

15. A, L. A. et al. MYC-containing amplicons in acute myeloid leukemia: genomic structures, evolution, and transcriptional consequences. Leukemia 32, 2152-2166, doi:10.1038/s41375-018-0033-0 (2018). 7

16. Baylin, S. B. & Jones, P. A. Epigenetic Determinants of Cancer. Cold Spring Haab Perspect Biol 8, doi: 10. 110 1/cshperspect.a019505 (2016).

17. Lee, D. Y., Hayes, J. J., Pruss, D. & Wolffe, A. P. A positive role for histone acetylation in transcription factor access to nucleosomal DNA. Cell 72, 73-84 (1993).

18. Luger, K., Mader, A. W., Richmond, R. K., Sargent, D. F. & Richmond, T. J. Crystal structure of the nucleosome core particle at 2.8 A resolution. Nature 389, 251-260, doi:10.1038/38444 (1997).

19. Smith, G. et al. c-Myc-induced extrachromosomal elements carry active chromatin. Neoplasia 5, 110-120, doi:10.1016/s1476-5586(03)80002-7 (2003).

20. Chen, X. et al. ATAC-see reveals the accessible genome by transposase-mediated imaging and sequencing. Nat Methods 13, 1013-1020, doi:10.1038/nmeth.4031 (2016).

21. Solovei, I. et al. Topology of double minutes (dmins) and homogeneously staining regions (HSRs) in nuclei of human neuroblastoma cell lines. Genes Chromosomes Cancer 29, 297-308 (2000).

22. Fang, R. et al. Mapping of long-range chromatin interactions by proximity ligationassisted ChIP-seq. Cell Res 26, 1345-1348, doi:10.1038/cr.2016.137 (2016).

23. Mumbach, M. R. et al. HiChIP: efficient and sensitive analysis of protein-directed genome architecture. Nat Methods 13, 919-922, doi:10.1038/nmeth.3999 (2016).

24. Rowley, M. J. & Corces, V. G. Organizational principles of 3D genome architecture. Nat Rev Genet 19, 789-800, doi:10.1038/s41576-018-0060-8 (2018).

25. Bailey, M. H. et al. Comprehensive Characterization of Cancer Driver Genes and Mutations. Cell 173, 371-385 e318, doi:10.1016/j.ce11.2018.02.060 (2018).

26. deCarvalho, A. C. et al. Discordant inheritance of chromosomal and extrachromosomal DNA elements contributes to dynamic disease evolution in glioblastoma. Nat Genet 50, 708-717, doi:10.1038/s41588-018-0105-0 (2018).

27. Lederberg, J. Cell genetics and hereditary symbiosis. Physiol Rev 32, 403-430, doi:10.1152/physrev.1952.32.4.403 (1952).

28. Nathanson, D. A. et al. Targeted therapy resistance mediated by dynamic regulation of extrachromosomal mutant EGFR DNA. Science 343, 72-76, doi:10.1126/science.1241328 (2014).

29. McGranahan, N. & Swanton, C. Clonal Heterogeneity and Tumor Evolution: Past, Present, and the Future. Cell 168, 613-628, doi:10.1016/j.ce11.2017.01.018 (2017).

30. Xu, K. et al. Structure and evolution of double minutes in diagnosis and relapse brain tumors. Acta Neuropathol 137, 123-137, doi:10.1007/s00401-018-1912-1 (2019).

31. Cao, H. et al. Rapid detection of structural variation in a human genome using nanochannel-based genome mapping technology. Gigascience 3, 34, doi:10.1186/2047-217X-3-34 (2014).

32. Corces, M. R. et al. An improved ATAC-seq protocol reduces background and enables interrogation of frozen tissues. Nat Methods 14, 959-962, doi:10.1038/nmeth.4396 (2017).

33. Juric, I. et al. MAPS: Model-based analysis of long-range chromatin interactions from PLAC-seq and HiChIP experiments. PLoS Comput Biol 15, e 1006982, doi:10.1371/journal.pcbi.1006982 (2019).

34. Raviram, R. et al. 4C-ker: A Method to Reproducibly Identify Genome-Wide Interactions Captured by 4C-Seq Experiments. PLoS Comput Biol 12, el004780, doi:10.1371/journal.pcbi.1004780 (2016).

35. Thakore, P. I. et al. Highly specific epigenome editing by CRISPR-Cas9 repressors for silencing of distal regulatory elements. Nat Methods 12, 1143-1149, doi:10.1038/nmeth.3630 (2015).

INFORMAL SEQUENCE LISTING

criEGFR#1
GGCTGGGCCTGCAAGTCCGCG (SEQ ID NO: 1)
criEGFR#2
GCACTTGGCACACTTGAACCA (SEQ ID NO: 2)
criNC (negative control)
GACGGAGGCTAAGCGTCGCAA (SEQ ID NO: 3)
Reading primer (4C-seq)
AATGATACGGCGACCACCGAGATCTACACACACTCTTTCCCTACACGAC
GCTCTTCCGATCTTTCCAAGAGCCAGGCCCGTAC (SEQ ID NO: 4)
Non-reading primer (4C-seq)
CAAGCAGAAGACGGCATACGAGATCACTGTGTGACTGGAGTTCAGACGT
GTGCTCTTCCGATCGAGCCCCATTTTGAAACACC (SEQ ID NO: 5)

What is claimed is:

1. A method of treating cancer in a subject in need thereof, wherein the cancer comprises an oncogene contained on a circular extrachromosomal DNA, comprising:
administering a transcriptional inhibitor of a gene contained on the circular extrachromosomal DNA, wherein the transcriptional inhibitor of the gene inhibits expression of the oncogene contained in circular extrachromosomal DNA.

2. The method of claim 1, wherein the gene is the oncogene.

3. The method of claim 1, wherein the gene is not the oncogene.

4. The method of claim 1 or 3, wherein the gene and the oncogene are not contained on the same circular extrachromosomal DNA molecule.

5. The method of any one of claims 1 to 4, wherein the oncogene is MYC, cyclin D1, CDK4, CDK6, MDM2, MDM4, ABL1, ABL2, AKT1, AKT2, ATF1, BCL11A, BCL2, BCL3, BCL6, BCR, BRCA2, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFR1OP, FRGR2, FUS, GOLGA5, GOPC, HMGA1, HMGA2, HRAS, IRF4, JUN, KIT, KRAS, LCK, LMO2, MAF, MAML2, MET, MITF, MLL, MPL, MYB, MYCL1, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SS18, TCL1A, TET2, TFG, TLX1, TPR, or USP6.

6. The method of any of claims 1-4, wherein the oncogene is EGFR, c-Myc, N-Myc, cyclin D1, ErbB2, CDK4, CDK6, BRAF, MDM2, MDM4, KRAS, or C-MET.

7. The method of any of claims 1-6, wherein said transcriptional inhibitor comprises an antisense nucleic acid, a siRNA, a microRNA, a ribonucleoprotein complex, a CRISPRi complex, or a small molecule.

8. The method of claim 7, wherein the transcriptional inhibitor is a small molecule and the small molecule comprises 8-Cl-Ado, actinomycin D, AT8319M, cordycepin, dinaciclib, flavopiridol, fludarabine, P276-00, R547, RGB-286638, Roscovitine, or SNS-0329.

9. The method of any of claims 1-8, further comprising determining whether the oncogene and/or the gene are contained in circular extrachromosomal DNA.

10. The method of any of claims 1-9, wherein the cancer is a leukemia, a lymphoma, a melanoma, a neuroendocrine tumor, a carcinoma, or a sarcoma.

11. A method of treating cancer in a subject in need thereof, wherein the cancer comprises an oncogene contained on a circular extrachromosomal DNA, comprising:
administering a transcriptional inhibitor of a first gene contained on the circular extrachromosomal DNA, wherein the transcriptional inhibitor of the first gene inhibits expression of the first gene and the oncogene contained in circular extrachromosomal DNA.

12. The method of claim 11, wherein the first gene and the oncogene are on the same circular extrachromosomal DNA molecule.

13. The method of claim 11 or 12, wherein the first gene is an oncogene.

14. The method of claim 13, wherein the oncogene is EGFR, c-Myc, N-Myc, cyclin D1, ErbB2, CDK4, CDK6, BRAF, MDM2, or MDM4.

15. The method of claim 11, wherein the first gene and the oncogene are the same gene.

16. The method of claim 11, wherein the first gene and the oncogene are the different genes.

17. The method of any one of claims 11-16, wherein the first gene and the oncogene are not within the same topologically associating domain (TAD).

18. The method of any one of claims 11-17, wherein the first gene comprises a promoter, the second gene comprises an enhancer, and the promoter of the first gene interacts with an enhancer of the second gene contained on the circular extrachromosomal DNA.

19. The method of any one of claims 11-18, wherein said transcriptional inhibitor comprises an antisense nucleic acid, a siRNA, a microRNA, a ribonucleoprotein complex, a CRISPRi complex, or a small molecule.

20. The method of claim 19, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

21. The method of any one of claims 11-18, wherein the transcriptional inhibitor of the first gene comprises a transcriptional repressor domain.

22. The method of claim 19, wherein the transcriptional repressor domain is a Kruppel associated box (KRAB) domain.

23. The method of claim 11, further comprising administering a plurality of transcriptional inhibitors of a plurality of genes contained on the circular extrachromosomal DNA.

24. The method of any one of claims 11-23, further comprising determining whether the first gene and/or the second gene are contained on circular extrachromosomal DNA.

25. The method of any one of claims 11-24, wherein the cancer is a leukemia, a lymphoma, a melanoma, a neuroendocrine tumor, a carcinoma, or a sarcoma.

26. A method of treating cancer in a subject in need thereof, wherein the cancer comprises an oncogene on a circular extrachromosomal DNA, comprising administering a therapeutically effective amount of an agent that decreases chromatin accessibility of the circular extrachromosomal DNA, thereby treating the cancer in the subject.

27. The method of claim 13, wherein the agent increases chromatin compaction of the circular extrachromosomal DNA.

28. The method of any one of claims 26-27, wherein said agent is an antisense nucleic acid, a siRNA, a microRNA, a ribonucleoprotein complex, a CRISPRi complex, or a small molecule.

29. The method of claim 28, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

30. The method of any one of claims 26-28, further comprising determining whether the oncogene is contained on circular extrachromosomal DNA.

31. The method of any one of claims 26-30, wherein the cancer is a leukemia, a lymphoma, a melanoma, a neuroendocrine tumor, a carcinoma, or a sarcoma.

32. A method of treating cancer in a subject in need thereof, wherein cancer cells in the subject comprise a first extrachromosomal oncogene forming part of a circular extrachromosomal DNA, the method comprising administering to said subject a therapeutically effective amount of a transcriptional inhibitor of a first gene forming part of the circular extrachromosomal DNA in the cancer cells of the subject, wherein the first gene and the first extrachromosomal oncogene do or do not form part of a same topologically associating domain (TAD).

33. The method of claim 32, further comprising inhibiting expression of a second extrachromosomal oncogene, wherein the first gene and the second extrachromosomal oncogene do not form part of the same topologically associating domain (TAD).

34. The method of claim 32 or 33, wherein the first gene is an oncogene.

35. The method of claim 34, wherein the first gene, the first extrachromosomal oncogene and the second extrachromosomal oncogene are independently different.

36. The method of claim 34, wherein the first gene, the first extrachromosomal oncogene and the second extrachromosomal oncogene are the same.

37. The method of claim 35, wherein said first gene, said first extrachromosomal oncogene and said second extrachromosomal oncogene are independently KRAS, C-MET, EGFR, c-Myc, N-Myc, cyclin D1, ErbB2, CDK4, CDK6, BRAF, MDM2, or MDM4.

38. The method of any one of claims 32-37, wherein said transcriptional inhibitor is a promoter inhibitor.

39. The method of claim 38, wherein said promoter inhibitor comprises an antisense nucleic acid, a siRNA, a microRNA, a CRISPRi complex, a ribonucleoprotein complex, or a small molecule.

40. The method of claim 39, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

41. The method of claim 38, wherein said promoter inhibitor comprises a transcriptional repressor domain.

42. The method of claim 41, wherein said transcriptional repressor domain is a Kruppel associated box (KRAB) domain.

43. The method of any one of claims 32-42, comprising administering an effective amount of a plurality of transcriptional inhibitors.

44. The method of claim 43, wherein said transcriptional inhibitors are independently different. 45 . The method of any one of claims 32-44, further comprising determining whether said first extrachromosomal oncogene is contained on circular extrachromosomal DNA.

46. The method of any one of claims 32-45, wherein the cancer is a leukemia, a lymphoma, a melanoma, a neuroendocrine tumor, a carcinoma, or a sarcoma.

47. A method of inhibiting expression of a gene in a subject, wherein the gene is contained on circular extrachromosomal DNA, comprising:
administering a transcriptional inhibitor of the gene to the subject, thereby inhibiting the expression of the gene.

48. The method of claim 47, wherein the gene is an oncogene.

49. The method of claim 48, wherein the oncogene is MYC, cyclin D1, CDK4, CDK6, MDM2, MDM4, ABL1, ABL2, AKT1, AKT2, ATF1, BCL11A, BCL2, BCL3, BCL6, BCR, BRCA2, BRAF, CARD11, CBLB, CBLC, CCND1, CCND2, CCND3, CDX2, CTNNB1, DDB2, DDIT3, DDX6, DEK, EGFR, ELK4, ERBB2, ETV4, ETV6, EVI1, EWSR1, FEV, FGFR1, FGFR1OP, FRGR2, FUS, GOLGA5, GOPC, HMGA1, HMGA2, HRAS, IRF4, JUN, KIT, KRAS, LCK, LMO2, MAF, MAML2, MET, MITF, MLL, MPL, MYB, MYCL1, MYCN, NCOA4, NFKB2, NRAS, NTRK1, NUP214, PAX8, PDGFB, PIK3CA, PIM1, PLAG1, PPARG, PTPN11, RAF1, REL, RET, ROS1, SMO, SS18, TCL1A, TET2, TFG, TLX1, TPR, or USP6.

50. The method of claim 48, wherein the oncogene is KRAS, C-MET, EGFR, c-Myc, N-Myc, cyclin D1, ErbB2, CDK4, CDK6, BRAF, MDM2, or MDM4.

51. The method of any of claims 47-50, wherein the transcriptional inhibitor comprises an antisense nucleic acid, a siRNA, a microRNA, a ribonucleoprotein complex, a CRISPRi complex, or a small molecule.

52. The method of claim 51, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

53. The method of any of claims 47-51, further comprising determining whether the gene is contained on circular extrachromosomal DNA.

54. A method of inhibiting expression of a first gene and a second gene in a subject, wherein the first gene and second gene are contained on circular extrachromosomal DNA, comprising:
administering a transcriptional inhibitor of the first gene to the subject, thereby inhibiting the expression of the first gene and the second gene.

55. The method of claim 54, wherein the first gene and the second gene are on the same circular extrachromosomal DNA molecule.

56. The method of claim 54 or 55, wherein the first gene is an oncogene, the second gene is an oncogene, or both the first gene and second gene are oncogenes.

57. The method of claim 56, wherein the oncogene is KRAS, C-MET, EGFR, c-Myc, N-Myc, cyclin D1, ErbB2, CDK4, CDK6, BRAF, MDM2, or MDM4.

58. The method of claim 54, wherein the first gene and the second gene are the same.

59. The method of claim 54, wherein the first gene and the second gene are different.

60. The method of any one of claims 54-58, wherein the first gene and the second gene are not within the same topologically associating domain (TAD).

61. The method of any one of claims 54-60, wherein the first gene comprises a promoter, the second gene comprises an enhancer, and the promoter of the first gene interacts with the enhancer of the second gene contained on the circular extrachromosomal DNA.

62. The method of any one of claims 54-61, wherein said transcriptional inhibitor comprises an antisense nucleic acid, a siRNA, a microRNA, a ribonucleoprotein complex, a CRISPRi complex, or a small molecule.

63. The method of claim 62, wherein the transcriptional inhibitor is a small molecule and the small molecule is 8-Cl-Ado, actinomycin D, AT8319M, cordycepin, dinaciclib, flavopiridol, fludarabine, P276-00, R547, RGB-286638, Roscovitine, or SNS-032.

64. The method of any one of claims 54-62, wherein the transcriptional inhibitor of the first gene comprises a transcriptional repressor domain.

65. The method of claim 64, wherein the transcriptional repressor domain is a Kruppel associated box (KRAB) domain.

66. The method of any one of claims 54-65, further comprising inhibiting expression of a third gene contained on the circular extrachromosomal DNA with the transcriptional inhibitor of the first gene.

67. The method of claim 66, wherein the third gene is an oncogene.

68. The method of claim 67, wherein the oncogene is KRAS, C-MET, EGFR, c-Myc, N-Myc, cyclin D1, ErbB2, CDK4, CDK6, BRAF, MDM2, or MDM4.

69. The method of claim 54, wherein the first gene, the second gene, and the third gene are the same.

70. The method of claim 54, wherein the first gene, the second gene, and the third gene are different.

71. The method of any one of claims 54-69, wherein the first gene and the third gene are not within the same topologically associating domain (TAD).

72. The method of any one of claims 54-71, wherein the first gene comprises a promoter, the third gene comprises an enhancer, and the promoter of the first gene interacts with the enhancer of the third gene contained on the circular extrachromosomal DNA.

73. The method of any one of claims 54-72, further comprising inhibiting expression of all the genes contained on the circular extrachromosomal DNA with the inhibitor of a promoter of the first gene.

74. The method of any one of claims 54-73, further comprising determining whether the first gene and/or the second gene are contained on the circular extrachromosomal DNA.

75. A transcriptional inhibitor of a gene contained in a circular extrachromosomal DNA for use in any of the methods of claims 1-74.
